(19) United States
(12) Patent Application Publication
(10) Pub. No.: US 2022/0119787 A1
(43) Pub. Date: Apr. 21, 2022

(54) VIRUS THERAPY

(71) Applicant: BIOMEDREX AB, STOCKHOLM (SE)

(72) Inventors: Theodore Anastasius PRAMER, Järfälla (SE); Martin EKENBERG, Brottby (SE); Corinne KAY, Biggleswade (GB)

(21) Appl. No.: 17/431,945

(22) PCT Filed: Feb. 20, 2020
(86) PCT No.: PCT/EP2020/054429

§ 371 Date: Aug. 18, 2021

(30) Foreign Application Priority Data
Feb. 20, 2019 (SE) 1950215-2
Feb. 20, 2019 (SE) 1950216-0
Feb. 20, 2019 (SE) 1950217-8
Feb. 20, 2019 (SE) 1950218-6

(51) Int. Cl. C12N 9/22; C12N 15/11; C12N 15/90

(52) U.S. Cl. CPC C12N 9/22; C12N 2310/20; C12N 15/907; C12N 15/11

(57) ABSTRACT

There is provided a protein-RNA complex where here the protein is selected from one of Cpf1 and CAS13 and where the protein is Cpf1 and the RNA is an RNA guide strand which comprises a sequence that is one of SEQ ID NO 1 to 40 less the 5′ TTTN motif of said SEQ ID NO 1-40, or the protein is CAS13 and the polynucleotide is an RNA guide strand that comprises a sequence that is complimentary to one of SEQ ID NO 41 to 80.

### FIELD OF TECHNOLOGY

This invention relates to CRISPR-related type therapies of infections, for example a virus infection.

### BACKGROUND

Endonucleases are enzymes that cleave polynucleotides. Clustered regular interspaced short palindromic repeat (CRISPR)-type proteins are endonucleases that use an RNA guide strand to cut DNA or RNA at specific sites. CRISPR-type proteins can be used for editing of DNA or for RNA knockdown. Cpf1 and Cas13 are two recently identified CRISPR type proteins. The use of Cpf1 for genome editing is described in Zetsche et al (Zetsche et al., 2015, Cell 163, 759-771). The use of Cas13 for knockdown has been reported in Abudayyeh et al (Abudayyeh et al (2017) Nature, 550 280-284.

Pathogens, such as virus, bacteria and eukaryotic parasites are still a major cause of suffering and death. There is a need for improved therapies for infections.

### SUMMARY OF INVENTION

In a first aspect of the intervention there is provided a protein-RNA complex where here the protein is selected from one of Cpf1 and CAS13 and where the protein is Cpf1 and the RNA is an RNA guide strand which comprises a sequence that is one of SEQ ID NO 1 to 40 less the 5′ TTTN motif of said SEQ ID NO 1-40, or the protein is CAS13 and the polynucleotide is an RNA guide strand that comprises a sequence that is complimentary to one of SEQ ID NO 41 to 80. In one embodiment the protein is Cpf1 and the sequence is SEQ ID NO 3, which targets the gag-pol gene of HIV.

These sequences are suitable for targeting HIV virus sequences with Cpf1 or Cas13. These sequences are suitable for selectively targeting virus DNA or RNA from HIV from most strains while avoiding patient sequences.

In one embodiment the protein is Cpf1 and the RNA is an RNA guide strand which comprises a sequence that is one of SEQ ID NO 1 to 40 less the 5′ TTTN motif of said SEQ ID NO 1-40. The use of Cpf1 protein for cutting DNA causes double strand brakes with overhangs, making it difficult to repair for the endogenous DNA repair systems, which cause the infected cells to trigger apoptosis, causing elimination of virus infected cells. Alternatively, important virus genes are destroyed.

In one embodiment the protein is CAS13 and the RNA is an RNA guide strand that comprises a sequence that is complimentary to one of SEQ ID NO 41 to 80. The use of CAS13 causes the destruction of virus RNA (such as mRNA) in virus infected cells.

In second aspect there is provided a protein—RNA complex for use in therapy, in particular for use in the treatment of an HIV infection.

In a third aspect of the invention there is provided a plasmid encoding a) the protein Cpf1 and an RNA guide strand which comprises a sequence that is one of SEQ ID NO 1 to 40 less the 5′ TTTN motif of said SEQ ID NO 1-40, or b) the protein is CAS13 and an RNA guide strand that comprises a sequence that is complimentary to one of SEQ ID NO 41 to 80, where the plasmid is adapted for expression of the protein and the transcription of the RNA guide strand in a mammalian cell.

There is also provided a therapeutically acceptable virus, that, when introduced into human cells, cause the expression of i) the protein Cpf1 and an RNA guide strand which comprises sequence that is one of SEQ ID NO 1 to 40 less the 5′ TTTN motif of said SEQ ID NO 1-40, or ii) the protein is CAS13 and an RNA guide strand that comprises a sequence that is complimentary to one of SEQ ID NO 41 to 80.

In a fourth aspect of the invention there is provided a pharmaceutical composition comprising a) protein-RNA complex according to the first aspect of the invention, or b) a plasmid according to the third aspect of the invention or c) two separate plasmids of which one encodes the protein Cpf1 and the other encodes an RNA guide strand which comprises sequence that is one of SEQ ID NO 1 to 40 less the 5′ TTTN motif of said SEQ ID NO 1-40, where the plasmids are adapted for expression of the protein and transcription of the RNA guide strand in a mammalian cell, or d) two separate plasmids of which one encodes the protein is CAS13 and the other encodes an RNA guide strand that comprises a sequence that is complimentary to one of SEQ ID NO 41 to 80, where the plasmids are adapted for expression of the protein and transcription of the RNA guide strand in a mammalian cell, or e) an RNA guide strand for Cpf1 comprising a sequence selected from one of SEQ ID NO 1-40 less the 5′TTTN motif, and a plasmid that encodes the protein Cpf1 and where the plasmid is adapted for expression of the protein in a mammalian cell; or, f) an RNA guide strand for Cas13 comprising a sequence that is complimentary to one of SEQ ID NO 41 to 80, and a plasmid that encodes the protein Cas13 where the plasmid is adapted for expression of the protein in a mammalian cell, or g) a therapeutically acceptable virus as described above.

In a fifth aspect of the invention there is provided a method of treatment of HIV comprising administering a protein-RNA complex according to the first aspect of the invention or a plasmid according to the third aspect of the invention or a pharmaceutical composition according to the fourth aspect of the invention, to patient in need thereof.

In a sixth aspect of the invention there is provided a method of causing double strand breaks in a HIV-infected cells comprising using a protein-RNA complex comprising the protein Cpf1 and an RNA guide strand which comprises a sequence that is one of SEQ ID NO 1 to 40 less the 5′ TTTN motif of said SEQ ID NO 1-40, where the method comprises introducing the protein-RNA complex, or means for expression of these, in a cell.

In a seventh aspect of the invention there is provided a method for RNA knock-down of HIV RNA in a HIV-infected cell comprising using a protein-RNA complex comprising the CAS13 protein and an RNA guide strand that comprises a sequence that is complimentary to one of SEQ ID NO 41 to 80, and where the method comprises introducing the protein-RNA complex, or means for expression of these, in a cell.

In an eight aspect of the invention there is provided a RNA polynucleotide with a length of at most 100 nucleotides, preferably from 40 to 44 nucleotides, the sequence comprising a Cpf1 handle sequence, for example the sequence UAAUUUCUACUCUUGUAGAU, and an RNA sequence selected from one of SEQ ID NO 1 to SEQ ID NO 40 less the 5′TTTN motif or an RNA polynucleotide with a length of at most 100 nucleotides comprising a CAS13 handle sequence, and a sequence that is complimentary to one of the sequences SEQ ID NO 41 to SEQ ID NO 80.

In a similar manner it is provided corresponding aspects of the invention for the treatment of hepatitis B infection, Herpes 1 infection and Herpes virus type 2 infection. Hence it is provided protein-RNA complexes, plasmids, viruses, formulations and polynucleotides for these as well. The sequence numbers for these aspects are as follows:

Hepatitis B: SEQ ID NO 101-140 (Table 3-Cpf1 sequences) and 141-180 (Table 4 CAS13 sequences).

Herpes type 1: SEQ ID NO 181-220 (table 5 Cpf1 sequences) and 221-260 (Table 6 CAS13 sequences)

Herpes type 2: SEQ ID NO 261-300 (table 7 Cpf1 sequences) and 301-341 (Table 8 CAS13 sequences).

Moreover, its should be noted that priority is claimed from four separate patent applications, one for each virus type, where the corresponding sequence numbers is as follows:

| | Corresponding sequence | Corresponding sequence |
|---|---|---|
| | numbers in priority | numbers in priority |
| | application for Cpf1 | application for CAS13 |
| | sequences. | sequences. |
| HIV | 1-40 | 41-80 |
| Hepatitis B | 1-40 | 41-80 |
| HSV1 | 1-40 | 41-80 |
| HSV 2 | 1-40 | 41-80 |

### BRIEF DESCRIPTION OF DRAWINGS

FIG. 1 is a schematic diagram showing an RNA guide strand.

### DETAILED DESCRIPTION

In brief, a recombinant CRISPR type protein in complex with an RNA guide strand (protein-RNA complex) that targets the CRISPR type protein to the DNA or the RNA of a pathogen is used for treating an infection in a patient. The protein-RNA complex specifically cuts polynucleotides of the pathogen that causes the infection. The patient may be a human or an animal, preferably a mammalian animal. In a preferred embodiment the patient is a human.

In a more general sense, the protein-RNA complex may be used to cause double strand breaks in the DNA of pathogen-infected cells, or to knock down RNA in a pathogen-infected cell. The protein-RNA complex may be directed to target a genomic locus of interest of the pathogen.

In a preferred embodiment the disease being treated is an infection caused by a pathogen such as a virus, a bacteria or a eukaryotic parasite, such as a fungus. In a preferred embodiment the infection is caused by a bacteria or a virus, most preferably a virus and most preferably a virus chosen from the group consisting of HIV (Human immunodeficiency Virus), HPV (Human Papilloma Virus), Herpes type 1, Herpes type 2 and Hepatitis B. In a preferred embodiment the virus infection being treated is a HIV infection, preferably HIV-1.

CRISPR-type proteins use an RNA guide strand to cut DNA or RNA. A guide strand is an RNA molecule that binds to the CRISPR-type protein and guides the CRISPR type protein to a certain polynucleotide target sequence. The guide strand is able to hybridize with the target strand (Watson-Crick base pairing). A complex between a CRISPR-type protein and an RNA guide strand is referred to as a protein-RNA complex herein.

As used herein “handle motif” and “handle sequence” refers to a RNA sequence that interacts with a CRISPR type protein for example by mediating binding between an RNA guide strand and a CRISPR type protein. Examples of handle sequences for Cpf1 and CAS13 are given below.

There are many different useful CRISPR-type proteins. Preferably the CRISPR-type protein has endonuclease activity that causes a double strand breaks. The most studied CRISPR protein is CRISPR/Cas9 which cuts DNA, leaving blunt ends. CRISPR/Cas9 has been used for editing of eukaryotic genomes (Cong et al, Science 339, (2013) 819-823, Mali et al, Science (2013) 823-826). CRISPR/Cas9 uses a 42-nucleotide RNA guide strand and in addition a second strand (so called tracrRNA strand) which may be 89 nucleotides long.

In a preferred embodiment, one of the CRISPR-type proteins Cpf1 or CAS13 is used. The use of Cpf1 for genome editing is described in Zetsche et al (Zetsche et al., 2015, Cell 163, 759-771). The use of CAS13 for RNA knockdown has been reported in Abudayyeh et al (Abudayyeh et al (2017) Nature, 550 280-284).

In contrast to CRISPR/Cas 9, Cpf1 (Zetsche et al., 2015, Cell 163, 759-771) cuts DNA in a staggered manner, leaving sticky ends with a 4 or 5 nucleotide 5′-overhang. This makes it difficult for DNA-repair system to repair the cut, compared to if a blunt end is created. The unligated DNA may inhibit the pathogen in different ways including but not limited to: 1) triggering apoptosis of a virus-infected cell, 2) causing the death of a pathogen, for example a bacterium. The pathogen is preferably a pathogen that has its genomic material in the form of DNA during at least some part of its life cycle. Many virus genomes become integrated into the host genomic DNA. For example, HIV becomes integrated into the genomic DNA of infected T-lymphocytes.

The Cpf1 protein may be a Cpf1 protein from Francisella novicida, Adamiococcus sp BV3L6 or Lachnospiracea bacterium ND2006 in particular Adamiococcus sp BV3L6 or Lachnospiracea bacterium ND2006. A useful variant of Cpf1 is Alt-R Cas12a.

CRISPR/Cas 13 cuts RNA and can be used for knockdown of pathogen RNA. This may limit pathogen survival, replication or activation, or may cause the death of the pathogen. The Cas13 protein may be Cas13 from Leptotrichia wadei (Abudayyeh et al (2017) Nature, 550 280-284. Useful variants of Cas13 include PspCas13b, LwaCas13a, LbuCas13a and LshCas13a, LwaCAS13 and PsmCAS13.

Other useful CRISPR type proteins edit polynucleotides by inserting an extra base in the polynucleotides, for example mRNA, leading to a frameshift and premature stop of translation.

When it is referred to CRISPR/Cas 9, Cpf1 and CAS13 it also includes functional equivalents and homologues of these proteins. Thus, modified or truncated proteins are included, provided that they have the same or comparable nuclease activity as the endogenous CRISPR/Cas 9, Cpf1 and CAS13 proteins. A homologue may have an amino acid identify with the original protein sequence of at least 70% more preferably at least 80%, even more preferably at least 90%, even more preferably at least 95% and most preferably at least 99%, using amino acid sequence alignment in BLAST (for example BLAST2 sequences) using the following settings: word size: 3, gapcosts: 11, 1, Matrix: BLOSUM62, Filter string: F, Window Size 40, Threshold 11.

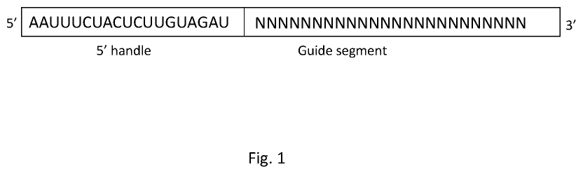

With reference to FIG. 1, the guide strand for Cpf1 preferably has a length of from 40 to 44, more preferably 41 to 44 nucleotides and comprises a 5′ constant motif (handle sequence) which may be 5′-AAUUUCUACUCUUGUAGAU-3′ or 5′-UAAUUUCUACUCUUGUAGAU-3′. The handle sequence interacts with Cpf1 and may be important for complexing with Cpf1 or for Cpf1 activity. The guide segment is 21-24 nucleotides long and is located 3′-terminal to the handle sequence. The RNA is provided as single-stranded RNA but parts, in particular parts of the handle sequence, may form a secondary structure. The guide segment of the guide strand hybridizes with a target strand of double stranded DNA. The opposite strand is referred to as the “displaced strand”.

Some CRISPR type proteins, including Cpf1, uses a PAM (Protospacer Adjacent Motif) motif to recognise target sequences. The minimal PAM motif for Cpf1 is TTN. The TTN motif used for Cpf1 is preferably TTT, even more preferably TTTV where V is any nucleotide except T. The PAM motif is localized on the displaced strand and is not recognized by the guide strand of the RNA-protein complex but by the interaction between the TTN nucleotides and amino acid residues of the Cpf1 protein. Cpf1 cuts the displaced strand with a 4-5 nucleotide overhang approximately 18-19 nucleotides from the TTN motif and cuts the target strand approximately 24-25 nucleotides from the TTN motif.

In addition, the guide strand for Cpf1 may have a unspecific 5′ extension of from 3 to 59 or more nucleotides in order to increase the delivery efficacy as described in Park et al., Nature Communications, (2018) 9:3313 DOI: 10.1038/s41467-018-05641-3. The 5′ extension is preferably not homologous to the human genome. For example, it may be a scrambled sequence. It has been hypothesized that such a 5′ extension increases efficacy by providing a negative charge.

For treatment of HIV in humans with a protein-RNA complex comprising Cpf1 protein, the protein-RNA complex is preferably directed to a sequence selected from SEQ ID NO 1 to SEQ ID NO 40, shown in Table 1. These sequences represent the displaced strands of various suitable targets. These sequences have the following properties: 1) they include the TTT PAM important for Cpf1 binding to the displaced strand, 2) the sequences are conserved over a large number of HIV stains, 3) the sequences are present in sequences that are important for the HIV virus, and 4) the sequences are not present in the human consensus genome, making it safe to target the protein-RNA complex to these sequence. These sequences ensure that the endonuclease activity of the protein-RNA complex will only be targeted to DNA in HIV-infected cells.

TABLE 1
HIV sequences for use with cpf1

| SEQ | | | | |
|---|---|---|---|---|
| ID | | | Coverage | Cut |
| NO | Sequence | Count | (%) | effiency |
| 1 | TTTAAAAGAAAAGGGGGGATTGGG | 2951 | 43.82 | 48.77 |
| 2 | TTTAAAAGAAAAGGGGGGACTGGA | 2876 | 42.71 | 61.99 |
| 3 | TTTCCCCTGCACTGTACCCCCCAA | 2709 | 40.23 | 70.16 |
| 4 | TTTGTATGTCTGTTGCTATTATGT | 2646 | 39.29 | 50.91 |
| 5 | TTTGACTAGCGGAGGCTAGAAGGA | 2641 | 39.22 | 71.23 |
| 6 | TTTATATAATTCACTTCTCCAATT | 2628 | 39.03 | 46.19 |
| 7 | TTTCGGGTTTATTACAGGGACAGC | 2611 | 38.77 | 46.34 |
| 8 | TTTATCTACTTGTTCATTTCCTCC | 2609 | 38.74 | 40.58 |
| 9 | TTTCTTCCAATTATGTTGACAGGT | 2583 | 38.36 | 35.14 |
| 10 | TTTATTTTTTCTTCTGTCAATGGC | 2564 | 38.08 | 1.24 |
| 11 | TTTGATAAAACCTCCAATTCCCCC | 2557 | 37.97 | 50.95 |
| 12 | TTTAGTTTGTATGTCTGTTGCTAT | 2516 | 37.36 | 49.94 |
| 13 | TTTATATTTATATAATTCACTTCT | 2512 | 37.3 | 19.13 |
| 14 | TTTACCCATGCATTTAAAGTTCTA | 2498 | 37.1 | 62.01 |
| 15 | TTTAAAATTGTGAATGAATACTGC | 2497 | 37.08 | 50.14 |
| 16 | TTTACTGGTACAGTTTCAATAGGA | 2488 | 36.95 | 80.49 |
| 17 | TTTAAATCTTGTGGGGTGGCTCCT | 2485 | 36.9 | 31.02 |
| 18 | TTTGAATTTTTGTAATTTGTTTTT | 2482 | 36.86 | 1.14 |
| 19 | TTTAATTTTACTGGTACAGTTTCA | 2475 | 36.75 | 40.14 |
| 20 | TTTCTTCTGTCAATGGCCATTGTT | 2419 | 35.92 | 54.76 |
| 21 | TTTCTTTTAAAATTGTGAATGAAT | 2399 | 35.63 | 10.99 |
| 22 | TTTGGGCCATCCATTCCTGGCTTT | 2340 | 34.75 | 48.26 |
| 23 | TTTCCACACAGGTACCCCATAATA | 2293 | 34.05 | 68.95 |
| 24 | TTTCAGGCCCAATTTTTGAAATTT | 2264 | 33.62 | 6.45 |
| 25 | TTTATCAAAGTAAGACAGTATGAT | 2241 | 33.28 | 43.03 |
| 26 | TTTCCATCCCTGTGGAAGCACATT | 2218 | 32.94 | 44.53 |
| 27 | TTTCCATAATCCCTAATGATCTTT | 2209 | 32.8 | 67.58 |
| 28 | TTTGCATGGCTGCTTGATGTCCCC | 2208 | 32.79 | 49.35 |
| 29 | TTTCCCTAAAAAATTAGCCTGTCT | 2198 | 32.64 | 76.35 |
| 30 | TTTGTTTTTGTAATTCTTTAGTTT | 2193 | 32.57 | 1.24 |
| 31 | TTTGTAATTCTTTAGTTTGTATGT | 2190 | 32.52 | 3.19 |
| 32 | TTTACTGGCCATCTTCCTGCTAAT | 2184 | 32.43 | 69.76 |
| 33 | TTTCCACATGTTAAAATTTTCTGT | 2183 | 32.42 | 21.46 |
| 34 | TTTGCTGGTCCTTTCCAAAGTGGA | 2167 | 32.18 | 43.18 |
| 35 | TTTGGGGTTGCTCTGGAAAACTCA | 2164 | 32.14 | 62.57 |
| 36 | TTTGTTCCTGAAGGGTACTAGTAG | 2156 | 32.02 | 44.52 |
| 37 | TTTGGATGGGTTATGAACTCCATC | 2142 | 31.81 | 62.52 |
| 38 | TTTGCTGTTGCACTATACCAGACA | 2138 | 31.75 | 75.22 |
| 39 | TTTCAAAAATTGGGCCTGAAAATC | 2137 | 31.73 | 34.47 |
| 40 | TTTAACATTTGCATGGCTGCTTGA | 2127 | 31.59 | 42.13 |

SEQ ID NO 1-40 show the sequences of the displaced strands, including the PAM motif (TTTV). The TTTV motif is not actually “displaced” by the protein-RNA complex, but remains hybridized to the target strand (Yamano et al 2016, Cell 165 949-962). For obtaining a suitable RNA guide sequence from SEQ ID NO 1-40 the following operations are performed, shown with SEQ ID NO 1 as an example (these operations can be done manually using pen and paper, word processing software or bioinformatics software):
1. Remove PAM.

SEQ ID NO 1 is:

5′- TTTAAAAGAAAAGGGGGGATTGGG -3′

SEQ ID NO 1 less the TTTN motif is:

(SEQ ID NO 82)
5′- AAAGAAAAGGGGGGATTGGG-3′.

2. Replace T with U:s:

Since RNA has U instead of T, the guide sequence strand is

(SEQ ID NO 83)
5′-AAAGAAAAGGGGGGAUUGGG-3′.

3. Add “handle sequence”

The guide sequence strand has a 5′ “handle” sequence that makes the guide RNA bind to the Cpf1 protein. The handle sequence may be 5′-UAAUUUCUACUCUUGUAGAU-3′ (SEQ ID NO 84).

Thus, the guide strand sequence is 5′-UAAUUUCUACUCUUGUAGAU-3′+5′-AAAGAAAAGGGGGGAUUGGG-3′

which is 5′-UAAUUUCUACUCUUGUAGAUAAAGAAAAGGGGGGAUUGGG-3′ (SEQ ID NO 85).

For use with Cpf1 from Acidaminococcus sp. the handle sequence may be 5′-AAUUUCUACUCUUGUAGAUG-3′ (SEQ ID NO 86).

Note that, because the TTN motif is on the opposite strand of the target strand, the guide strand will comprise one of SEQ ID 1-40. The target sequence will be the reverse complement of each of SEQ ID NO 1-40.

Examples of suitable target RNA sequences for targeting CAS13 to HIV RNA, for example HIV mRNA, include SEQ ID NO 41-80, shown in Table 2.

These sequences have the following properties: 1) the sequences are conserved over a large number of HIV stains, 2) the sequences are present in sequences that are important for the HIV virus, and 3) the sequences are not present in the human consensus genome, making it safe to target the protein-RNA complex to these sequences. Protein-RNA complexes with these RNA guide strands cuts crucial HIV mRNA.

TABLE 2
HIV sequences for use with CAS13

| SEQ ID | | | Coverage | Cut |
|---|---|---|---|---|
| NO | Sequence | Count | (%) | effiency |
| 41 | CACAAUUUUAAAAGAAAAGGGGGGAUUGGG | 2941 | 43.67 | 88.98 |
| 42 | ACAAUUUUAAAAGAAAAGGGGGGAUUGGGG | 2936 | 43.6 | 88.43 |
| 43 | CAAUUUUAAAAGAAAAGGGGGGAUUGGGGG | 2871 | 42.63 | 80.77 |
| 44 | UAUGGAAAACAGAUGGCAGGUGAUGAUUGU | 2814 | 41.79 | 68.53 |
| 45 | GAAAGGUGAAGGGGCAGUAGUAAUACAAGA | 2800 | 41.58 | 66.65 |
| 46 | GGAAAGGUGAAGGGGCAGUAGUAAUACAAG | 2800 | 41.58 | 66.73 |
| 47 | UGGAAAGGUGAAGGGGCAGUAGUAAUACAA | 2800 | 41.58 | 67.68 |
| 48 | AAAAGGGGGGAUUGGGGGGUACAGUGCAGG | 2797 | 41.54 | 71.34 |
| 49 | GAAAAGGGGGGAUUGGGGGGUACAGUGCAG | 2796 | 41.52 | 71.76 |
| 50 | AGAAAAGGGGGGAUUGGGGGGUACAGUGCA | 2795 | 41.51 | 71.74 |
| 51 | CUCUGGAAAGGUGAAGGGGCAGUAGUAAUA | 2795 | 41.51 | 68.67 |
| 52 | CUGGAAAGGUGAAGGGGCAGUAGUAAUACA | 2794 | 41.49 | 69.23 |
| 53 | UCUGGAAAGGUGAAGGGGCAGUAGUAAUAC | 2794 | 41.49 | 68.50 |
| 54 | AAACAGAUGGCAGGUGAUGAUUGUGUGGCA | 2784 | 41.34 | 69.12 |
| 55 | AGGGGGGAUUGGGGGGUACAGUGCAGGGGA | 2782 | 41.31 | 71.42 |
| 56 | AAAACAGAUGGCAGGUGAUGAUUGUGUGGC | 2778 | 41.25 | 65.28 |
| 57 | GAAAACAGAUGGCAGGUGAUGAUUGUGUGG | 2777 | 41.24 | 65.25 |
| 58 | GGAAAACAGAUGGCAGGUGAUGAUUGUGUG | 2777 | 41.24 | 64.51 |
| 59 | AUGGAAAACAGAUGGCAGGUGAUGAUUGUG | 2777 | 41.24 | 65.26 |
| 60 | UGGAAAACAGAUGGCAGGUGAUGAUUGUGU | 2777 | 41.24 | 65.24 |
| 61 | AAUUUUAAAAGAAAAGGGGGGAUUGGGGGG | 2762 | 41.02 | 71.24 |
| 62 | AUUUUAAAAGAAAAGGGGGGAUUGGGGGGU | 2761 | 41 | 70.52 |
| 63 | UUUUAAAAGAAAAGGGGGGAUUGGGGGGUA | 2758 | 40.96 | 71.05 |
| 64 | AAAAAUUCAAAAUUUUCGGGUUUAUUACAG | 2758 | 40.96 | 78.76 |
| 65 | UAGAAGGAGAGAGAUGGGUGCGAGAGCGUC | 2749 | 40.82 | 55.79 |
| 66 | CUAGAAGGAGAGAGAUGGGUGCGAGAGCGU | 2746 | 40.78 | 56.36 |
| 67 | GCUAGAAGGAGAGAGAUGGGUGCGAGAGCG | 2741 | 40.7 | 56.82 |
| 68 | GGCUAGAAGGAGAGAGAUGGGUGCGAGAGC | 2738 | 40.66 | 56.64 |
| 69 | AAAGGGGGGAUUGGGGGGUACAGUGCAGGG | 2728 | 40.51 | 68.54 |
| 70 | AAGGGGGGAUUGGGGGGUACAGUGCAGGGG | 2727 | 40.5 | 68.51 |
| 71 | UUAAAAGAAAAGGGGGGAUUGGGGGGUACA | 2723 | 40.44 | 69.08 |
| 72 | UUUAAAAGAAAAGGGGGGAUUGGGGGGUAC | 2723 | 40.44 | 69.23 |
| 73 | UCUUGGGAGCAGCAGGAAGCACUAUGGGCG | 2722 | 40.42 | 46.53 |
| 74 | AGGCUAGAAGGAGAGAGAUGGGUGCGAGAG | 2720 | 40.39 | 55.94 |
| 75 | UUCUUGGGAGCAGCAGGAAGCACUAUGGGC | 2720 | 40.39 | 44.83 |
| 76 | AUUAUGGAAAACAGAUGGCAGGUGAUGAUU | 2719 | 40.38 | 61.74 |
| 77 | CUUGGGAGCAGCAGGAAGCACUAUGGGCGC | 2717 | 40.35 | 46.28 |
| 78 | UUAUGGAAAACAGAUGGCAGGUGAUGAUUG | 2716 | 40.33 | 62.43 |
| 79 | AAAAGAAAAGGGGGGAUUGGGGGGUACAGU | 2713 | 40.29 | 67.84 |
| 80 | UAAAAGAAAAGGGGGGAUUGGGGGGUACAG | 2712 | 40.27 | 67.18 |

Similar operations as described above for Cpf1 can be done with SEQ ID NO 41-80. However, because these sequences are targeting by CAS13 which targets RNA, the guide strand will comprise a sequence that is the reverse complement of one of SEQ ID 41-80.

SEQ ID NO 41 will be used as an example:

SEQ ID NO 41 is 5′-CACAAUUUUAAAAGAAAAGGGGGGAUUGGG-3′. The guide sequence will be the reverse complement of this sequence, which is

(SEQ ID NO 88)
5′- CCCAAUCCCCCCUUUUCUUUUAAAAUUGUG - 3′

The guide strand comprises a so called “direct repeat sequence” (DRS) (“handle sequence”) that is specific for the CAS13 protein used, and which interacts with the CAS13 protein, and may mediate binding of the guide sequence to the CAS13 protein and CAS13 activity. For some CAS13 proteins, the DRS is located 5′ of the guide segment and for others the DRS is located 3′ of the guide segment.

Below are some examples of DRS for CAS 13 proteins:

Prevotella sp. (Psp) Cas13b: 5′-GUUGUGGAAGGUCCAGUUUUGAGGGGCUAUUACAAC-3′ (located 3′ of guide segment) (SEQ ID NO 89)

Lepterotrichia shahii (Lwa) Cas13a: 5′-GAUUUAGACUACCCCAAAAACGAAGGGGACUAAAAC-3′ (located 5′ of the guide segment) (Freije et al, 2019, Molecular Cell 76, 826-837) (SEQ ID NO 90)

Thus, a suitable guide strand sequence for the Lwa CAS13 protein for targeting SEQ ID NO 41 may be 5′-GAUUUAGACUACCCCAAAAACGAAGGGGACUAAAAC-3′

+
5′- CCCAAUCCCCCCUUUUCUUUUAAAAUUGUG - 3′

which is

(SEQ ID NO 91)
5′- GAUUUAGACUACCCCAAAAACGAAGGGGACUAAAAC
CCCAAUCCCCCCUUUUCUUUUAAAAUUGUG - 3′

The guide strands for some Cas13 proteins (but not Cas13a from Lwa or CAS13b from Psp) may in addition need a protospacer flanking site (PFS), se for example Abudayyeh et al (2017) Nature, 550 280-284 where the PFS for Leptotrichia shahii Cas13a is discussed. The PFS may be a preference for H (=not G). It is referred to (Smargon, Cox, Pyzocha et al., Molecular cell 2017; Cox, Gootenberg, Abudayyeh et al., Science 2017).

The guide strand for CAS13 preferably has a length of less than 100 nucleotides. Preferably the length is from 50 to 100 nucleotides more preferably from 60 to 80 nucleotides.

In a similar way there is provided sequences that can be used in the treatment of Hepatitis B virus, Herpes type 1 virus and Herpes type 2 virus, as shown in Tables 3-8, below.

TABLE 3
Hepatitis sequences for use with Cpf1.

| SEQ | | | | |
|---|---|---|---|---|
| ID | | | Coverage | Cut |
| NO | Sequence | Count | (%) | effiency |
| 101 | TTTACTAGTGCCATTTGTTCAGTG | 6230 | 90.58 | 48.43 |
| 102 | TTTGTTCAGTGGTTCGTAGGGCTT | 5839 | 84.89 | 23.74 |
| 103 | TTTCTTTTGTCTTTGGGTATACAT | 5293 | 76.96 | 3.84 |
| 104 | TTTGAAGTATGCCTCAAGGTCGGT | 5208 | 75.72 | 39.89 |
| 105 | TTTCTCGCCAACTTACAAGGCCTT | 5146 | 74.82 | 44.77 |
| 106 | TTTCAGTTATATGGATGATGTGGT | 5043 | 73.32 | 67.90 |
| 107 | TTTCCGGAAGTGTTGATAAGATAG | 4878 | 70.92 | 48.49 |
| 108 | TTTGCCTTCTGACTTCTTTCCTTC | 4761 | 69.22 | 51.64 |
| 109 | TTTGGAGTGTGGATTCGCACTCCT | 4629 | 67.3 | 64.95 |
| 110 | TTTATGCCTACAGCCTCCTAGTAC | 4451 | 64.71 | 82.68 |
| 111 | TTTGTACTAGGAGGCTGTAGGCAT | 4367 | 63.49 | 47.30 |
| 112 | TTTCCCCCACTGTTTGGCTTTCAG | 4255 | 61.86 | 40.43 |
| 113 | TTTCTTGTTGACAAGAATCCTCAC | 4246 | 61.73 | 64.49 |
| 114 | TTTGGTGTCTTTTGGAGTGTGGAT | 4148 | 60.31 | 23.36 |
| 115 | TTTGGGGTGGAGCCCTCAGGCTCA | 4140 | 60.19 | 66.78 |
| 116 | TTTGGAGCTTCTGTGGAGTTACTC | 3835 | 55.76 | 58.48 |
| 117 | TTTAAATGTATACCCAAAGACAAA | 3666 | 53.3 | 98.91 |
| 118 | TTTGTCTTTGGGTATACATTTAAA | 3666 | 53.3 | 32.15 |
| 119 | TTTGTGGCTCCTCTGCCGATCCAT | 3652 | 53.1 | 34.04 |
| 120 | TTTACGTCCCGTCGGCGCTGAATC | 3649 | 53.05 | 42.47 |
| 121 | TTTCTCCTGGCTCAGTTTACTAGT | 3617 | 52.59 | 57.63 |
| 122 | TTTGTTTACGTCCCGTCGGCGCTG | 3604 | 52.4 | 11.63 |
| 123 | TTTGTGGGTCACCATATTCTTGGG | 3599 | 52.33 | 43.37 |
| 124 | TTTGGAAGACCAACCTCCCATGCT | 3575 | 51.98 | 85.90 |
| 125 | TTTCACATTTCCTGTCTTACTTTT | 3539 | 51.45 | 44.82 |
| 126 | TTTACGCGGTCTCCCCGTCTGTGC | 3498 | 50.86 | 46.57 |
| 127 | TTTCACCTCTGCCTAATCATCTCA | 3482 | 50.63 | 79.52 |
| 128 | TTTCACTTTCTCGCCAACTTACAA | 3470 | 50.45 | 46.18 |
| 129 | TTTGGCTTTCAGTTATATGGATGA | 3281 | 47.7 | 61.62 |
| 130 | TTTCCATGGCTGCTAGGCTGTGCT | 3272 | 47.57 | 59.25 |
| 131 | TTTATCATATTCCTCTTCATCCTG | 2913 | 42.35 | 43.98 |
| 132 | TTTATACGGGTCAATGTCCATGCC | 2879 | 41.86 | 41.72 |
| 133 | TTTGCTGCCCCTTTTACACAATGT | 2865 | 41.65 | 28.31 |
| 134 | TTTACACAGAAAGGCCTTGTAAGT | 2822 | 41.03 | 53.46 |
| 135 | TTTACGCGGACTCCCCGTCTGTGC | 2779 | 40.4 | 54.37 |
| 136 | TTTGTCCTGGTTATCGCTGGATGT | 2777 | 40.38 | 42.40 |
| 137 | TTTATTCTTCTACTGTACCTGTCT | 2694 | 39.17 | 39.06 |
| 138 | TTTCACCTCTGCCTAATCATCTCT | 2654 | 38.59 | 78.50 |
| 139 | TTTCTCTTGGCTCAGTTTACTAGT | 2636 | 38.33 | 54.67 |
| 140 | TTTCCTGTCTTACTTTTGGAAGAG | 2629 | 38.22 | 39.14 |

TABLE 4
Hepatitis sequences for use with CAS13

| | | | Coverage | Cut |
|---|---|---|---|---|
| SEQ ID NO | Sequence | Count | (%) | effiency |
| 141 | CCGUGUGCACUUCGCUUCACCUCUGCACGU | 6657 | 96.79 | 71.85 |
| 142 | AGAGUCUAGACUCGUGGUGGACUUCUCUCA | 6586 | 95.75 | 79.58 |
| 143 | CAGAGUCUAGACUCGUGGUGGACUUCUCUC | 6492 | 94.39 | 77.47 |
| 144 | UUCAAGCCUCCAAGCUGUGCCUUGGGUGGC | 6488 | 94.33 | 73.76 |
| 145 | CAAGCCUCCAAGCUGUGCCUUGGGUGGCUU | 6488 | 94.33 | 73.03 |
| 146 | UCAAGCCUCCAAGCUGUGCCUUGGGUGGCU | 6487 | 94.32 | 73.75 |
| 147 | AAGCCUCCAAGCUGUGCCUUGGGUGGCUUU | 6485 | 94.29 | 73.72 |
| 148 | ACUCGUGGUGGACUUCUCUCAAUUUUCUAG | 6402 | 93.08 | 76.88 |
| 149 | CGUGGUGGACUUCUCUCAAUUUUCUAGGGG | 6399 | 93.04 | 77.57 |
| 150 | UCGUGGUGGACUUCUCUCAAUUUUCUAGGG | 6397 | 93.01 | 77.54 |
| 151 | CUCGUGGUGGACUUCUCUCAAUUUUCUAGG | 6397 | 93.01 | 76.81 |
| 152 | GACUCGUGGUGGACUUCUCUCAAUUUUCUA | 6397 | 93.01 | 77.54 |
| 153 | CUAGACUCGUGGUGGACUUCUCUCAAUUUU | 6395 | 92.98 | 77.51 |
| 154 | UAGACUCGUGGUGGACUUCUCUCAAUUUUC | 6392 | 92.93 | 77.47 |
| 155 | UCUAGACUCGUGGUGGACUUCUCUCAAUUU | 6376 | 92.7 | 77.23 |
| 156 | AGACUCGUGGUGGACUUCUCUCAAUUUUCU | 6374 | 92.67 | 77.21 |
| 157 | AGUCUAGACUCGUGGUGGACUUCUCUCAAU | 6368 | 92.59 | 77.12 |
| 158 | GUCUAGACUCGUGGUGGACUUCUCUCAAUU | 6366 | 92.56 | 77.09 |
| 159 | GAGUCUAGACUCGUGGUGGACUUCUCUCAA | 6361 | 92.48 | 77.01 |
| 160 | UGCAACUUUUUCACCUCUGCCUAAUCAUCU | 6313 | 91.79 | 71.20 |
| 161 | GUUCAAGCCUCCAAGCUGUGCCUUGGGUGG | 6310 | 91.74 | 71.15 |
| 162 | UGUUCAAGCCUCCAAGCUGUGCCUUGGGUG | 6303 | 91.64 | 71.05 |
| 163 | ACUGUUCAAGCCUCCAAGCUGUGCCUUGGG | 6296 | 91.54 | 70.95 |
| 164 | CUGUUCAAGCCUCCAAGCUGUGCCUUGGGU | 6292 | 91.48 | 70.89 |
| 165 | UAGAAGAAGAACUCCCUCGCCUCGCAGACG | 6274 | 91.22 | 79.39 |
| 166 | CAUGCAACUUUUUCACCUCUGCCUAAUCAU | 6266 | 91.1 | 71.24 |
| 167 | AUGCAACUUUUUCACCUCUGCCUAAUCAUC | 6255 | 90.94 | 71.08 |
| 168 | CUAGAAGAAGAACUCCCUCGCCUCGCAGAC | 6252 | 90.9 | 79.07 |
| 169 | CCUAGAAGAAGAACUCCCUCGCCUCGCAGA | 6218 | 90.4 | 77.84 |
| 170 | CCCUAGAAGAAGAACUCCCUCGCCUCGCAG | 6213 | 90.33 | 77.77 |
| 171 | CCCCUAGAAGAAGAACUCCCUCGCCUCGCA | 6207 | 90.24 | 77.68 |
| 172 | UCAGUUUACUAGUGCCAUUUGUUCAGUGGU | 6187 | 89.95 | 65.70 |
| 173 | UGGCUCAGUUUACUAGUGCCAUUUGUUCAG | 6178 | 89.82 | 65.56 |
| 174 | GGCUCAGUUUACUAGUGCCAUUUGUUCAGU | 6176 | 89.79 | 65.53 |
| 175 | GUUUACUAGUGCCAUUUGUUCAGUGGUUCG | 6176 | 89.79 | 66.27 |
| 176 | AGUUUACUAGUGCCAUUUGUUCAGUGGUUC | 6175 | 89.78 | 66.25 |
| 177 | CAGUUUACUAGUGCCAUUUGUUCAGUGGUU | 6175 | 89.78 | 65.52 |
| 178 | CUCAGUUUACUAGUGCCAUUUGUUCAGUGG | 6172 | 89.74 | 65.48 |
| 179 | GCUCAGUUUACUAGUGCCAUUUGUUCAGUG | 6170 | 89.71 | 65.45 |
| 180 | CCAUGCAACUUUUUCACCUCUGCCUAAUCA | 6085 | 88.47 | 68.59 |

TABLE 5
Herpes type 1 sequences for use with Cpf1.

| SEQ | | | | |
|---|---|---|---|---|
| ID | | | Coverage | Cut |
| NO | Sequence | Count | (%) | effiency |
| 181 | TTTAATAAAAATAACCAAAAACAC | 19 | 90.48 | 67.03 |
| 182 | TTTGTTTGAAATGTTTTGTTTTTA | 19 | 90.48 | 1.26 |
| 183 | TTTGGGTGGGTGGGGAGTGGGTGG | 19 | 90.48 | 46.01 |
| 184 | TTTGAAATGTTTTGTTTTTATTGT | 19 | 90.48 | 1.22 |
| 185 | TTTAGGATGCCAGCCAGGGCGGCG | 17 | 80.95 | 45.73 |
| 186 | TTTATACACAGATGTCAACGCCGC | 17 | 80.95 | 53.94 |
| 187 | TTTGGAAATAGCAACAAGGCCGTG | 17 | 80.95 | 60.28 |
| 188 | TTTGGATGGTATGGTCCAGATGCT | 17 | 80.95 | 81.49 |
| 189 | TTTCTGACCTGCACCGATCGCAGC | 17 | 80.95 | 60.88 |
| 190 | TTTAGTTCTATGATGACACAAACC | 17 | 80.95 | 63.43 |
| 191 | TTTGGCGTACATGTTTTGGGCCGC | 17 | 80.95 | 25.54 |
| 192 | TTTCTGTTTCTTTAACCCGTCTGG | 17 | 80.95 | 19.73 |
| 193 | TTTAGGCCGCAACCCGGCCGAGCC | 17 | 80.95 | 66.97 |
| 194 | TTTCGGTACAGCAGGTAGAGACAC | 17 | 80.95 | 101.15 |
| 195 | TTTGAGCAGATGTTTACCGATGCC | 17 | 80.95 | 52.37 |
| 196 | TTTCTACCTCCCCGGGGCCTGCAT | 17 | 80.95 | 42.73 |
| 197 | TTTGCTTCCCCGGACTTCGCGCCC | 17 | 80.95 | 45.35 |
| 198 | TTTGACTCAGACGCAGGGCCCGGG | 17 | 80.95 | 27.32 |
| 199 | TTTAAAAAGATATACAGTAAGACA | 17 | 80.95 | 55.77 |
| 200 | TTTCACTGGGAGCGCTTTCCGGAC | 17 | 80.95 | 61.39 |
| 201 | TTTGGGTAAACACCTTTAATAAGC | 17 | 80.95 | 71.24 |
| 202 | TTTAATTACCATACCGGGAAGTGG | 17 | 80.95 | 47.89 |
| 203 | TTTGCCGGGACGGTGACCGGGCGA | 17 | 80.95 | 51.21 |
| 204 | TTTACGGATCGGTGTATAAATTAC | 17 | 80.95 | 64.71 |
| 205 | TTTGGGGCGTGTCTGTTTCTTGGC | 17 | 80.95 | 35.45 |
| 206 | TTTCTGTCGTCGGAGGCCCCCGGG | 17 | 80.95 | 30.69 |
| 207 | TTTGGGCCGCAATGCGCGTGGCGC | 17 | 80.95 | 22.64 |
| 208 | TTTGGCCTGACGGAAAGGCTTCGC | 17 | 80.95 | 57.10 |
| 209 | TTTGACTTGCACGAACTCGCTGAC | 17 | 80.95 | 63.71 |
| 210 | TTTGGGGATTGGCGGCCAGGCCCG | 17 | 80.95 | 28.87 |
| 211 | TTTGCCAGCAACCTGACCGCGCTG | 17 | 80.95 | 57.53 |
| 212 | TTTGAAACTGACATCGCGATACCC | 17 | 80.95 | 37.70 |
| 213 | TTTGCATCGGAGCGCACGCGGGAA | 17 | 80.95 | 48.08 |
| 214 | TTTCTGGATGGCCGACATTTCCCC | 17 | 80.95 | 59.95 |
| 215 | TTTCGTTTTCTCCCCCGAAGTCAG | 17 | 80.95 | 47.76 |
| 216 | TTTGGGCGCCTCCGGATCACCAAC | 17 | 80.95 | 69.00 |
| 217 | TTTCCTCATGGCCCCTTTTATACC | 17 | 80.95 | 31.09 |
| 218 | TTTGGGGAGGGGAAAGGCGTGGGG | 17 | 80.95 | 37.56 |
| 219 | TTTGCGTGGCCGCCTCGTAAAACC | 17 | 80.95 | 55.79 |
| 220 | TTTAAAAAGGCCTCGGCCCTCCCT | 17 | 80.95 | 54.28 |

TABLE 6
Herpes type 1 sequences for use with CAS13.

| | | | Coverage | Cut |
|---|---|---|---|---|
| SEQ ID NO | Sequence | Count | (%) | effiency |
| 221 | UGGGUGGGGAGUGGGUGGGUGGGGAGUGGG | 19 | 90.48 | 61.84 |
| 222 | UUUUUGGGUGGGUGGGGAGUGGGUGGGUGG | 19 | 90.48 | 63.30 |
| 223 | AGUGGGUGGGUGGGGAGUGGGUGGGUGGGG | 19 | 90.48 | 58.92 |
| 224 | UUGAUUUUUGGGUGGGUGGGGAGUGGGUGG | 19 | 90.48 | 63.30 |
| 225 | UGAUUUUUGGGUGGGUGGGGAGUGGGUGGG | 19 | 90.48 | 63.30 |
| 226 | UUUUGGGUGGGUGGGGAGUGGGUGGGUGGG | 19 | 90.48 | 63.30 |
| 227 | GAGUGGGUGGGUGGGGAGUGGGUGGGUGGG | 19 | 90.48 | 58.92 |
| 228 | AUAAUGUAAUUGGUGGAUGAGAAGUAGGUG | 19 | 90.48 | 74.26 |
| 229 | GGUGGGUGGGGAGUGGGUGGGUGGGGAGUG | 19 | 90.48 | 61.84 |
| 230 | UUGGGUGGGUGGGGAGUGGGUGGGUGGGGA | 19 | 90.48 | 63.30 |
| 231 | GUGGGUGGGGAGUGGGUGGGUGGGGAGUGG | 19 | 90.48 | 61.84 |
| 232 | UAAUGUAAUUGGUGGAUGAGAAGUAGGUGA | 19 | 90.48 | 74.99 |
| 233 | UGGGUGGGUGGGGAGUGGGUGGGUGGGGAG | 19 | 90.48 | 63.30 |
| 234 | UUUGGGUGGGUGGGGAGUGGGUGGGUGGGG | 19 | 90.48 | 63.30 |
| 235 | GGAGUGGGUGGGUGGGGAGUGGGUGGGUGG | 19 | 90.48 | 59.65 |
| 236 | AUUUUUGGGUGGGUGGGGAGUGGGUGGGUG | 19 | 90.48 | 63.30 |
| 237 | GGGAGUGGGUGGGUGGGGAGUGGGUGGGUG | 19 | 90.48 | 60.38 |
| 238 | GAUUUUUGGGUGGGUGGGGAGUGGGUGGGU | 19 | 90.48 | 63.30 |
| 239 | GGUGGGGAGUGGGUGGGUGGGGAGUGGGUG | 19 | 90.48 | 61.11 |
| 240 | GGGGAGUGGGUGGGUGGGGAGUGGGUGGGU | 19 | 90.48 | 61.11 |
| 241 | GGGUGGGUGGGGAGUGGGUGGGUGGGGAGU | 19 | 90.48 | 62.57 |
| 242 | GGGUGGGGAGUGGGUGGGUGGGGAGUGGGU | 19 | 90.48 | 61.11 |
| 243 | GGUUGAUUUUUGGGUGGGUGGGGAGUGGGU | 19 | 90.48 | 63.30 |
| 244 | GUGGGGAGUGGGUGGGUGGGGAGUGGGUGG | 19 | 90.48 | 60.38 |
| 245 | GUGGGUGGGUGGGGAGUGGGUGGGUGGGGA | 19 | 90.48 | 58.92 |
| 246 | GUUGAUUUUUGGGUGGGUGGGGAGUGGGUG | 19 | 90.48 | 63.30 |
| 247 | UGGGGAGUGGGUGGGUGGGGAGUGGGUGGG | 19 | 90.48 | 60.38 |
| 248 | GGGGGGGAGAGGGGAGAGGGGGGGAGAGGG | 18 | 85.71 | 68.00 |
| 249 | GGGGGAGAGGGGAGAGGGGGGGAGAGGGGA | 18 | 85.71 | 66.54 |
| 250 | GGGGAGAGGGGGGGAGAGGGGAGAGGGGGG | 18 | 85.71 | 68.00 |
| 251 | GGGGGGAGAGGGGAGAGGGGGGGAGAGGGG | 18 | 85.71 | 68.00 |
| 252 | GGGAGAGGGGGGGAGAGGGGAGAGGGGGGG | 18 | 85.71 | 68.00 |
| 253 | GAGGGGGGGAGAGGGGAGAGGGGGGGAGAG | 18 | 85.71 | 68.73 |
| 254 | AGGGGAGAGGGGGGGAGAGGGGAGAGGGGG | 18 | 85.71 | 68.00 |
| 255 | AGAGGGGGGGAGAGGGGAGAGGGGGGGAGA | 18 | 85.71 | 68.00 |
| 256 | AGGGGGGGAGAGGGGAGAGGGGGGGAGAGG | 18 | 85.71 | 68.73 |
| 257 | GGAGAGGGGGGGAGAGGGGAGAGGGGGGGA | 18 | 85.71 | 68.00 |
| 258 | GAGAGGGGGGGAGAGGGGAGAGGGGGGGAG | 18 | 85.71 | 68.00 |
| 259 | GGGGAGAGGGGAGAGGGGGGGAGAGGGGAG | 18 | 85.71 | 67.27 |
| 260 | AAAAAAGGGAGGGACGGGGGCCGGCAGACC | 17 | 80.95 | 56.62 |

TABLE 7
Herpes type 2 sequences for use with Cpf1.

| SEQ | | | | |
|---|---|---|---|---|
| ID | | | Coverage | Cut |
| NO | Sequence | Count | (%) | effiency |
| 261 | TTTCTCCTTCCTCTTCCCTTCCAC | 43 | 100 | 51.90 |
| 262 | TTTATATTTATAAAAATTTTACAA | 43 | 100 | 1.28 |
| 263 | TTTATTGTAAAATTTTTATAAATA | 43 | 100 | 2.06 |
| 264 | TTTACCCTCACCCCACCCCATCCT | 41 | 95.35 | 98.88 |
| 265 | TTTATTATTAATTACACCAACCAC | 41 | 95.35 | 35.26 |
| 266 | TTTCATATATTTTAAATAAACAAA | 41 | 95.35 | 30.20 |
| 267 | TTTGTTTATTTAAAATATATGAAA | 41 | 95.35 | 1.33 |
| 268 | TTTATAATTCTTTTTTATTTCCCA | 41 | 95.35 | 1.23 |
| 269 | TTTATACACAACACCAACCTTTCC | 41 | 95.35 | 71.78 |
| 270 | TTTCCACCCCCCTTCCCCCTCCTT | 41 | 95.35 | 50.12 |
| 271 | TTTATTTTATACACAACACCAACC | 41 | 95.35 | 24.73 |
| 272 | TTTGTGTTTATTTAAGGAGAAGGG | 40 | 93.02 | 8.33 |
| 273 | TTTGTTGGATGATGGATTGATTGA | 40 | 93.02 | 46.24 |
| 274 | TTTGGGGGGGGTGTTTGGGTGGGA | 40 | 93.02 | 42.53 |
| 275 | TTTCTGGCCTTGTTGAAAACTTGA | 39 | 90.7 | 27.36 |
| 276 | TTTGACGGCGCCGGGGTTGAAGCG | 39 | 90.7 | 28.61 |
| 277 | TTTATCGCCGCGGCTGCGCCCTGC | 39 | 90.7 | 26.23 |
| 278 | TTTATGAAGTACGTCGAGTGGTCG | 39 | 90.7 | 32.43 |
| 279 | TTTGGCCCCCACCAGAGCGAGTGG | 39 | 90.7 | 50.86 |
| 280 | TTTAACTTTGGGGATTTCGGCGAC | 39 | 90.7 | 32.76 |
| 281 | TTTCCTGGGTGTCGGCCGGAAACA | 39 | 90.7 | 67.54 |
| 282 | TTTCACGTAGGCGAACATGCTGTC | 39 | 90.7 | 85.35 |
| 283 | TTTCGGGGCCTCGACAAGGAGGCG | 39 | 90.7 | 38.43 |
| 284 | TTTGTTTCGTGCGTTTGGGTGCGG | 39 | 90.7 | 6.97 |
| 285 | TTTAATTACCATAAGCGGGAATGG | 39 | 90.7 | 48.07 |
| 286 | TTTCCGGAGTTAGCAAAACCGATA | 39 | 90.7 | 56.06 |
| 287 | TTTCCGCCCTCCTCCCTCCCCACC | 39 | 90.7 | 56.47 |
| 288 | TTTACCAGCGCCCCCTCGCCGACG | 39 | 90.7 | 69.46 |
| 289 | TTTCGAGACGGGCGCATGTCCAAG | 39 | 90.7 | 55.87 |
| 290 | TTTATCGGTTGGACGCGTTTCCCT | 39 | 90.7 | 38.28 |
| 291 | TTTCCGCGCGGCTGGGTGAGCGTG | 39 | 90.7 | 36.55 |
| 292 | TTTCCTGGCCCTCAAGCAGGGGCC | 39 | 90.7 | 53.12 |
| 293 | TTTCCCCCGCCTCCCGTCTTCTTC | 39 | 90.7 | 70.40 |
| 294 | TTTCACGGCGACGATCCGTTTGGG | 39 | 90.7 | 42.88 |
| 295 | TTTCATTATCTTCGCCCTGGAGCA | 39 | 90.7 | 53.65 |
| 296 | TTTCGCCGAACCCCATGGCCTCGA | 39 | 90.7 | 77.36 |
| 297 | TTTGTTGTATGTAACCTGACCGGA | 39 | 90.7 | 54.00 |
| 298 | TTTGGTCCTTTTCTCTGGTTTCGG | 39 | 90.7 | 9.01 |
| 299 | TTTATACGCGGACCCCAGCACCAC | 39 | 90.7 | 49.14 |
| 300 | TTTCCAAGCGGGTGGTGATGTTTG | 39 | 90.7 | 38.68 |

TABLE 8
Herpes type 2 sequences for use with CAS13

| | | | Coverage | Cut |
|---|---|---|---|---|
| SEQ ID NO | Sequence | Count | (%) | effiency |
| 301 | GUGGGUGGAAGGGAAGAGGAAGGAGAAAGG | 43 | 100 | 89.71 |
| 302 | GGUGGGUGGAAGGGAAGAGGAAGGAGAAAG | 43 | 100 | 89.71 |
| 303 | UGUAAAAUUUUUAUAAAUAUAAAGUUUUUU | 43 | 100 | 98.48 |
| 304 | GGUGGAAGGGAAGAGGAAGGAGAAAGGGGG | 43 | 100 | 91.17 |
| 305 | UGGGUGGAAGGGAAGAGGAAGGAGAAAGGG | 43 | 100 | 90.44 |
| 306 | GUAAAAUUUUUAUAAAUAUAAAGUUUUUUU | 43 | 100 | 98.48 |
| 307 | GGGGGAAGAGAGGGGGAGGUAGGGAGGGGA | 43 | 100 | 83.86 |
| 308 | GAAGAGAGGGGGAGGUAGGGAGGGGAGAGG | 43 | 100 | 82.40 |
| 309 | UUGUAAAAUUUUUAUAAAUAUAAAGUUUUU | 43 | 100 | 98.48 |
| 310 | GGGUGGAAGGGAAGAGGAAGGAGAAAGGGG | 43 | 100 | 91.17 |
| 311 | GUGGAAGGGAAGAGGAAGGAGAAAGGGGGG | 43 | 100 | 91.90 |
| 312 | UAUUGUAAAAUUUUUAUAAAUAUAAAGUUU | 43 | 100 | 99.21 |
| 313 | UUAUUGUAAAAUUUUUAUAAAUAUAAAGUU | 43 | 100 | 99.21 |
| 314 | UAAAAUUUUUAUAAAUAUAAAGUUUUUUUU | 43 | 100 | 98.48 |
| 315 | UUUAUUGUAAAAUUUUUAUAAAUAUAAAGU | 43 | 100 | 99.21 |
| 316 | GGAAGAGAGGGGGAGGUAGGGAGGGGAGAG | 43 | 100 | 82.40 |
| 317 | GGGAAGAGAGGGGGAGGUAGGGAGGGGAGA | 43 | 100 | 83.13 |
| 318 | GGGGAAGAGAGGGGGAGGUAGGGAGGGGAG | 43 | 100 | 83.13 |
| 319 | GGGUGGGUGGAAGGGAAGAGGAAGGAGAAA | 43 | 100 | 88.98 |
| 320 | AUUGUAAAAUUUUUAUAAAUAUAAAGUUUU | 43 | 100 | 98.48 |
| 321 | GGGGGUGGGUGGAAGGGAAGAGGAAGGAGA | 43 | 100 | 87.51 |
| 322 | GGGGUGGGUGGAAGGGAAGAGGAAGGAGAA | 43 | 100 | 88.24 |
| 323 | UGAUGUAAUUGGUGGAUGAGAAGUAGGUGA | 41 | 95.35 | 79.90 |
| 324 | UGGGAGGUGGGUGUUUGUAUGUGUGGGAGA | 41 | 95.35 | 75.52 |
| 325 | AUGAUGUAAUUGGUGGAUGAGAAGUAGGUG | 41 | 95.35 | 79.17 |
| 326 | UGUUGGAUGAUGGAUUGAUUGAUUUUAUUG | 40 | 93.02 | 73.17 |
| 327 | UAUUUUGUUGGAUGAUGGAUUGAUUGAUUU | 40 | 93.02 | 71.71 |
| 328 | GUUGGAUGAUGGAUUGAUUGAUUUUAUUGA | 40 | 93.02 | 73.91 |
| 329 | UUAUUUUGUUGGAUGAUGGAUUGAUUGAUU | 40 | 93.02 | 71.71 |
| 330 | GGGGGAGGUAGGGAGGGGAGAGGAGAAGGG | 40 | 93.02 | 75.37 |
| 331 | GGGGGAGGAAAAAGAAUAAAGGGGGUAGUG | 40 | 93.02 | 73.17 |
| 332 | GGGGGGAGGAAAAAGAAUAAAGGGGGUAGU | 40 | 93.02 | 73.17 |
| 333 | GAUAUGUGAGUUUGGUUGUGUUUUGUGGGA | 40 | 93.02 | 80.48 |
| 334 | GAGAGGGGGAGGUAGGGAGGGGAGAGGAGA | 40 | 93.02 | 76.10 |
| 335 | GAGGGGGAGGUAGGGAGGGGAGAGGAGAAG | 40 | 93.02 | 75.37 |
| 336 | AGGGGGAGGUAGGGAGGGGAGAGGAGAAGG | 40 | 93.02 | 75.37 |
| 337 | AGAGAGGGGGAGGUAGGGAGGGGAGAGGAG | 40 | 93.02 | 75.37 |
| 338 | AAGAGAGGGGGAGGUAGGGAGGGGAGAGGA | 40 | 93.02 | 75.37 |
| 339 | AGAGGGGGAGGUAGGGAGGGGAGAGGAGAA | 40 | 93.02 | 76.10 |
| 340 | AUUUUGUUGGAUGAUGGAUUGAUUGAUUUU | 40 | 93.02 | 72.44 |

For use with Cpf1 the following sequences may be preferred:
SEQ ID 101 targets a Hepatitis B reading frame common for the P and S genes of the Hepatitis B genome.
SEQ ID NO 188 targets the UL20 and UL19 genes of Herpes type 1 virus genome.
SEQ ID NO 269 targets the UL29 gene of herpes type 2 virus genome.

In certain embodiments the guide segment may comprise a sequence that is similar or highly similar to one of SEQ ID NO 1-80 and 101-341, such that one or more of the nucleotides of SEQ ID NO 1-80 and 101-341 are replaced with a different nucleotide while maintaining the activity. Hence one of the nucleotides A, U, G, C are replaced by a different nucleotide. Because of the length of the guide segment the guide segment may still hybridize to the target strand. The number or substitutions may be 3, more preferably 2, and most preferably only one.

The protein-RNA complexes, or the plasmids or the virus are preferably administered to the patient in the form of a pharmaceutical composition. Such a pharmaceutical composition comprises an effective amount of the protein-RNA complexes, plasmids or virus (“active component”), and a pharmaceutically acceptable carrier, which typically is an aqueous solution optionally comprising a variety of different pharmacologically acceptable compounds. The formulation is made to suit the mode of administration. There is a wide variety of possible formulations. The formulation may be adapted to increase the uptake or stability of the active component or to improve the pharmacokinetics or pharmacodynamics of the active component, or to enhance other desirable properties of the formulation. The pharmaceutical composition, the complexes and the virus and plasmids described herein are preferably non-naturally occurring or engineered.

In certain embodiments a protein-RNA complex is delivered. Delivery of the protein-RNA complex can be made in any suitable way. Two reviews that describe useful methods of delivery are: Glass, Lee, LI and Xu; Trends in Biotechnology, 2017, and Liu, Zhang, Liu and Cheng, Journal of Controlled Disease, 266 (2017) 17-26.

Suitable methods include nanoparticles for example gold particles, or polymeric carriers, such as polymers obtained from chitosan or poly-caprolactone or poly-lactic/glycolic acid-copolymers. The use of gold particles is a preferred method of delivery (Mout et al (2017) ACS Nano 11, 2452-2458) and Lee et al Nature Biomedical Engineering volume 1, pages 889-901 (2017).

Another preferred method of delivery is lipid nanoparticles, for example as described in Wang et al., PNAS Mar. 15, 2016 vol. 113 no. 11 2868-2873, and Li et al., Biomaterials 178 (2018) 652-662.

In other embodiments, a plasmid or plasmids encoding the protein and/or the guide RNA is administered to the patient, as is known in the art. The plasmids are preferably adapted for expression of the protein and transcription of the RNA in the cell type of interest which may be a mammalian cell, preferably a human cell. For example, the protein gene and the guide strand gene is preferably under control of suitable promotors that induce expression in these cells. A skilled person knows how to achieve expression in mammalian cells. For plasmid delivery, the route of administration, formulation and dose can be as in U.S. Pat. No. 5,846,946 and as in clinical studies involving plasmids. In some embodiments, the guide strand is delivered (as RNA) together with a plasmid that encodes the CRISPR-type protein, or the other way around. When the plasmid or plasmids are delivered to pathogens that are bacterial or eukaryotes for expression in the pathogen, the promotor is preferably chosen to suit the internal transcription system of the pathogen. When the pathogen is a virus that has infected a human a suitable promotor for expression in humans may be chosen. For some viruses that have their own polymerases, promoters may be chosen to suit those polymerases.

In other embodiments delivery of the CRISPR type protein or the RNA guide strand is carried out with the use of a virus. The virus is preferably therapeutically acceptable, meaning that the virus method of delivery has a low intrinsic risk for the patient. The CRISPR-type protein and the guide RNA can be delivered using adeno associated virus (AAV), lentivirus, adenovirus or other plasmid or viral vector types, in particular, using formulations and doses from, for example, U.S. Pat. No. 8,454,972 (formulations, doses for adenovirus), 8,404,658 (formulations, doses for AAV) and 5,846,946 (formulations, doses for DNA plasmids) and from clinical trials and publications regarding the clinical trials involving lentivirus, AAV and adenovirus. For examples, for AAV, the route of administration, formulation and dose can be as in U.S. Pat. No. 8,454,972 and as in clinical trials involving AAV. For adenovirus, the route of administration, formulation and dose can be as in U.S. Pat. No. 8,404,658 and as in clinical trials involving adenovirus.

When a virus or a plasmid is used, each of the sequences encoding the CRISPR-type protein and so the guide strand is adapted for expression of the protein in the cell, and adapted for transcription of RNA. Thus, the coding sequences are preferably under control of a regulatory element, which typically is a DNA sequence that controls the transcription of the gene of interest. The regulatory element may comprise one more promotors, enhancers or the like. The regulatory element is chosen to suit the cell in which expression is to be achieved. The regulatory element may be operably linked to the sequences. Each of the CRISPR-type protein and the sequence encoding the guide stand may be operably linked to a separate regulatory element. The genes for the CRISPR-type protein may be codon-optimized for expression in the cells of the interest, for example human cells. The CRISPR-type protein and/or the guide strand may be targeted to the nucleus with the addition of nucleus targeting sequences.

Multiple guide strands that each target one separate sequence may be delivered simultaneously, for example with the use of a plasmid that encodes a plurality of guide strands or with one long RNA that is broken up into a plurality of guide strands with the use of a nuclease activity.

The formulation may be adapted for parenteral administration such as for example intraarticular, intravenous, intradermal, intraperitoneal, or subcutaneous administration, and may include aqueous and non-aqueous injection solutions. Formulations for injection may be in unit dosage forms, for example ampules, or in multidosage forms. The formulation can be for administration topically, systemically or locally. The formulation can also be provided as an aerosol.

The formulations may contain nuclease inhibitors (such as RNase inhibitors) antioxidants, buffers, antibiotics, salts, solutes that renders the formulation isotonic, lipids, carriers, diluents, emulsifiers, chelating agents, excipients, fillers, drying agents, antioxidants, binding agents, solubilizers, stabilizers, antimicrobial agents, preservatives, and the like.

The protein-RNA complex, the plasmids or the virus may be administered to the subject in any suitable manner. The protein-RNA complexes, the plasmids or the virus can be administered by a number of routes including intravenous injection, intraperitoneal, intramuscular, transdermal, subcutaneous, topical, sublingual, or rectal means. Suitable modes of administration include injection or infusion. Intravenous administration is a preferred mode of administration.

Preferably an effective amount of the protein-RNA complex, the plasmids or the virus is administered to the subject. An effective amount is an amount that is able to treat one or more symptoms of a disease, halt or reverse the progression of a disease.

Administration may be carried out at a single time point or repeatedly over a time period or from an implanted slow-release matrix. Other delivery systems include bolus injections, time-release, delayed release, sustained release or controlled release systems.

Dosage and administration regimens may be determined by methods known in the art, for example with testing in appropriate in vitro or in vivo models, such as animal models in order to analyse efficacy, pharmacokinetics, pharmacodynamics, excretion, tissue uptake and the like, by methods known in the art. A suitable way of finding a suitable dose is starting with a low amount and gradually increasing the amount.

The above-mentioned methods for administration of protein-RNA complexes, plasmids and virus can be used to introduce double strand breaks with the use of Cpf1 in virus-infected cells or pathogen (such as bacteria)-infected cells or to knock down pathogen (such as viral) RNA using CAS13, in vivo, ex vivo or in vitro. In one embodiment this is done in vitro. The pathogen-infected cells may be a subpopulation of a larger population of cells, where not all cells are infected with the pathogenic virus.

There are known in vitro methods for assessing the efficacy of treatment and delivery. Examples include the methods used in Ueda et at, Microbiology and Immunology Volume 60, Issue 7, July 2016, 483-496.

The CRISPR type protein for use in protein-RNA complexes are preferably produced in a suitable expression system. Production of protein with the use of expression systems is well known in the art. In general, Current protocols in Molecular Biology (John Wiley & sons) provides guidance for polynucleotide handling and manipulation, and protein expression and handling. CRISPR type protein, in particular Cpf1 and CAS13 can be produced in any suitable manner. Suitable expression systems include eukaryotic cells such as CHO cells, insect cells or bacteria. Often, E. coli is the preferred expression system because of its ease of use, and because the CRISPR-type proteins are of bacterial origin. Typically, the production of protein involves cloning of the coding sequence for the protein into a plasmid suitable for expression. The plasmid preferably has a promotor that drives expression. For expression in E. coli the T7 promotor may be useful. For expression in mammalian cells, the CMV promotor may be useful. The plasmid is introduced into the cells with the use of well-known transfection protocols, and stable or transient expressing cells are generated. Suitable transfection techniques may be the use of electroporation or the use of liposomes, such as Lipofectamine® or virus-based methods. Clones stably expression the protein may be selected, expanded and propagated.

Expression plasmids for Cpf1 are described in Zetsche et al and expression plasmids for CAS13 are described in Abudayyeh et al (see above). The proteins may be expressed with a suitable tag for purification of the protein, such as poly-His tag.

Useful plasmids for expression of Cpf1 include pTE4396, pTE4396, pAsCpf1(TYCV)(BB) (pY211) and pY010 (pcDNA3.1-hAsCpf1). Useful plasmids for expression of Cas13 include: pC0046-EF1a-PspCas13b-NES-HIV and pC0056-LwCas13a-msfGFP-NES (eukaryotic expression) and p2CT-HisMBP-Lwa_Cas13a_WT (expression in bacteria).

Purification of protein may be carried out as is known in the art and may include steps such as: cell lysis, centrifugation, gel filtration, affinity chromatography and dialysis. The protein is preferably purified and endotoxin-free.

The RNA guide strand can be produced in any suitable way. A preferred way is chemical synthesis. Methods for synthesis of RNA are well known to a person skilled in the art. RNA synthesis is preferable done in a controlled environment to avoid degradation of RNA by for example RNAses. Synthesis may for example be carried out by adding and covalently attaching one base at a time to growing RNA chain. Examples of useful RNA synthesis machines include Oligo Synthesizer 192 from Oligomaker APS and ABI 3900 from Biolytic Lab Performance Inc. WO200364026 describes a useful polynucleotide synthesis machine. Alternatively, the guide strand can be expressed and purified from host cells.

The conditions for complexing guide RNA with protein are known. Typically, the protein is incubated with the guide RNA in a suitable buffer. Incubation time may be 10 minutes to 30 minutes. The guide strand will then bind to the protein.

### Example 1—HIV

Genomes for a large number of HIV subtypes (approx 3000 subtypes) where downloaded from www.hiv.lanl.ov. Data was imported into a table in a relational database.

### Example 2—HIV

An algorithm was used to search in the database of Example 1 for target sequences that that comprise a PAM sequence for Cpf1. Target sequences were scored for how many of the HIV virus genomes that have them. The most conserved sequences were selected for further processing. It was checked that none of the selected sequences were present in the consensus human genome. The sequences were then selected based on that they should be present in sequences that are important for virus survival, replication or activation. The results are shown in Table 1. The score shows percentage of strains that carry the sequence.

### Example 3—HIV

An algorithm was used to search in the database of sequences that are likely to be transcribed to RNA, as being suitable targets for CAS13. Target sequences were scored for how many of the HIV virus genomes that have them. The most conserved sequences were selected for further processing. It was checked that none of the selected sequences were present in the consensus human genome. The sequences were then selected based on that they should be present in sequences that are important for virus survival, replication or activation. The results are shown in Table 2. The score shows percentage of strains that carry the sequence.

### Example 4—HIV

Cpf1 protein will be produced as in Zetsche et al. Forty different RNA guide strands for targeting each of SEQ ID NO 1-40 are synthesized. Each guide strand consists of the 5′ handle sequence UAAUUUCUACUCUUGUAGAU followed by each of SEQ ID NO 1 to 40 less the 5′ TTTN motif.

RNA-protein complexes with each of the RNA guide strands and Cpf1 protein is formed. Gold particles are formed as described in Lee et al Nature Biomedical Engineering volume 1, pages 889-901 (2017). Each of the forty different complexes is tested in a suitable in vitro model for example the model used in Ueda et at, Microbiology and Immunology Volume 60, Issue 7, July 2016, 483-496

### Example 5—HIV

CAS13 protein will be produced as in Abudayyeh et al. Forty different guide RNA that comprises sequences complimentary to each of sequences SEQ ID NO 41 to 80 are synthesized. RNA-protein complexes with each of the RNA guide strands and CAS13 protein is formed. Gold particles are formed as described in Lee et al Nature Biomedical Engineering volume 1, pages 889-901 (2017).

The gold particles with the RNA protein complexes are provided to HIV infected T-lymphocytes in culture. Each of the forty different complexes is tested in suitable in vitro model, for example the model used in Ueda et at, Microbiology and Immunology Volume 60, Issue 7, July 2016, 483-496

While the invention has been described with reference to specific exemplary embodiments, the description is in general only intended to illustrate the inventive concept and should not be taken as limiting the scope of the invention. The invention is generally defined by the claims.

### Example 6—Hepatitis B

Genomes for a large number of hepatitis B subtypes where downloaded from a database. Data was imported into a table in a relational database.

### Example 7—Hepatitis B

An algorithm was used to search in the database of Example 1 for target sequences that that comprise a PAM sequence for Cpf1. Target sequences were scored for how many of the hepatitis B virus genomes that have them. The most conserved sequences were selected for further processing. It was checked that none of the selected sequences were present in the consensus human genome. The sequences were then selected based on that they should be present in sequences that are important for virus survival, replication or activation. The results are shown in Table 3. The score shows percentage of strains that carry the sequence.

### Example 8—Hepatitis B

An algorithm was used to search in the database of sequences that are likely to be transcribed to RNA, as being suitable targets for CAS13. Target sequences were scored for how many of the hepatitis B virus genomes that have them. The most conserved sequences were selected for further processing. It was checked that none of the selected sequences were present in the consensus human genome. The sequences were then selected based on that they should be present in sequences that are important for virus survival, replication or activation. The results are shown in Table 4. The score shows percentage of strains that carry the sequence.

### Example 9—Herpes Type 1

Genomes for a large number of HSV1 subtypes where downloaded from a database. Data was imported into a table in a relational database.

### Example 10—Herpes Type 1

An algorithm was used to search in the database of Example 1 for target sequences that that comprise a PAM sequence for Cpf1. Target sequences were scored for how many of the HSV1 virus genomes that have them. The most conserved sequences were selected for further processing. It was checked that none of the selected sequences were present in the consensus human genome. The sequences were then selected based on that they should be present in sequences that are important for virus survival, replication or activation. The results are shown in Table 5. The score shows percentage of strains that carry the sequence.

### Example 11—Herpes Type 1

An algorithm was used to search in the database of sequences that are likely to be transcribed to RNA, as being suitable targets for CAS13. Target sequences were scored for how many of the HSV1 virus genomes that have them. The most conserved sequences were selected for further processing. It was checked that none of the selected sequences were present in the consensus human genome. The sequences were then selected based on that they should be present in sequences that are important for virus survival, replication or activation. The results are shown in Table 6. The score shows percentage of strains that carry the sequence.

### Example 12—Herpes Type 2

Genomes for a large number of HSV2 subtypes where downloaded from a database. Data was imported into a table in a relational database.

### Example 13—Herpes Type 2

An algorithm was used to search in the database of Example 1 for target sequences that that comprise a PAM sequence for Cpf1. Target sequences were scored for how many of the HSV2 virus genomes that have them. The most conserved sequences were selected for further processing. It was checked that none of the selected sequences were present in the consensus human genome. The sequences were then selected based on that they should be present in sequences that are important for virus survival, replication or activation. The results are shown in Table 7. The score shows percentage of strains that carry the sequence.

### Example 14—Herpes Type 2

An algorithm was used to search in the database of sequences that are likely to be transcribed to RNA, as being suitable targets for CAS13. Target sequences were scored for how many of the HSV2 virus genomes that have them. The most conserved sequences were selected for further processing. It was checked that none of the selected sequences were present in the consensus human genome. The sequences were then selected based on that they should be present in sequences that are important for virus survival, replication or activation. The results are shown in Table 8. The score shows percentage of strains that carry the sequence.

While the invention has been described with reference to specific exemplary embodiments, the description is in general only intended to illustrate the inventive concept and should not be taken as limiting the scope of the invention. The invention is generally defined by the claims. For example, the methods of delivering the guide strand and the proteins are examples only. Any suitable method for delivery the sequences and the cpf1 and CAS13 proteins can be used.

1. A protein-RNA complex, wherein the protein is selected from one of Cpf1 and CAS13 and wherein
the protein is Cpf1 and the RNA is an RNA guide strand which comprises a sequence that is one of SEQ ID NO 1 to 40 less the 5′ TTTN motif of said SEQ ID NO 1-40, or
the protein is CAS13 and the polynucleotide is an RNA guide strand that comprises a sequence that is complimentary to one of SEQ ID NO 41 to 80.

2. The protein-RNA complex according to claim 1 where the protein is Cpf1 and the RNA is an RNA guide strand which comprises a sequence that is one of SEQ ID NO 1 to 40 less the 5′ TTTN motif of said SEQ ID NO 1-40.

3. The protein RNA complex according to claim 2 where the SEQ ID NO is SEQ ID NO 3.

4. The protein-RNA complex according to claim 1, wherein the protein is CAS13 and the RNA is an RNA guide strand that comprises a sequence that is complimentary to one of SEQ ID NO 41 to 80.

5. A protein-RNA complex according to claim 1 for use in therapy.

6. A protein-RNA complex according to claim 1 for use in the treatment of an HIV infection.

7. A plasmid encoding
a) the protein Cpf1 and an RNA guide strand which comprises a sequence that is one of SEQ ID NO 1 to 40 less the 5′ TTTN motif of said SEQ ID NO 1-40, or
b) the protein CAS13 and an RNA guide strand that comprises a sequence that is complimentary to one of SEQ ID NO 41 to 80,
where the plasmid is adapted for expression of the protein and the transcription of the RNA guide strand in a mammalian cell.

8. A therapeutically acceptable virus, that, when introduced into human cells, causes the expression of i) the protein Cpf1 and an RNA guide strand which comprises a sequence that is one of SEQ ID NO 1 to 40 less the 5′ TTTN motif of said SEQ ID NO 1-40, or ii) the protein CAS13 and an RNA guide strand that comprises a sequence that is complimentary to one of SEQ ID NO 41 to 80.

9. A pharmaceutical composition comprising
a. a protein-RNA complex according to claim 1, or
b. a plasmid encoding i) the protein Cpf1 and an RNA guide strand which comprises a sequence that is one of SEQ ID NO 1 to 40 less the 5′ TTTN motif of said SEQ ID NO 1-40, or ii) the protein CAS13 and an RNA guide strand that comprises a sequence that is complimentary to one of SEQ ID NO 41 to 80, where the plasmid is adapted for expression of the protein and the transcription of the RNA guide strand in a mammalian cell; or
c. two separate plasmids of which one encodes the protein Cpf1 and the other encodes an RNA guide strand which comprises a sequence that is one of SEQ ID NO 1 to 40 less the 5′ TTTN motif of said SEQ ID NO 1-40, where the plasmids are adapted for expression of the protein and transcription of the RNA guide strand in a mammalian cell, or
d. two separate plasmids of which one encodes the protein CAS13 and the other encodes an RNA guide strand that comprises a sequence that is complimentary to one of SEQ ID NO 41 to 80, where the plasmids are adapted for expression of the protein and transcription of the RNA guide strand in a mammalian cell, or
e. an RNA guide strand for Cpf1 comprising a sequence selected from one of SEQ ID NO 1-40 less the 5′TTTN motif, and a plasmid that encodes the protein Cpf1 and where the plasmid is adapted for expression of the protein in a mammalian cell; or
f. an RNA guide strand for Cas13 comprising a sequence that is complimentary to one of SEQ ID NO 41 to 80, and a plasmid that encodes the protein Cas13 where the plasmid is adapted for expression of the protein in a mammalian cell; or
g. a therapeutically acceptable virus, that, when introduced into human cells, causes the expression of i) the protein Cpf1 and an RNA guide strand which comprises a sequence that is one of SEQ ID NO 1 to 40 less the 5′ TTTN motif of said SEQ ID NO 1-40, or ii) the protein CAS13 and an RNA guide strand that comprises a sequence that is complimentary to one of SEQ ID NO 41 to 80.

10. A method of treatment of HIV infection comprising administering a protein-RNA complex, wherein the protein is selected from one of Cpf1 and CAS13 and wherein the protein is Cpf1 and the RNA is an RNA guide strand which comprises a sequence that is one of SEQ ID NO 1 to 40 less the 5′ TTTN motif of said SEQ ID NO 1-40, or the protein is CAS13 and the polynucleotide is an RNA guide strand that comprises a sequence that is complimentary to one of SEQ ID NO 41 to 80; or
a plasmid encoding i) the protein Cpf1 and an RNA guide strand which comprises a sequence that is one of SEQ ID NO 1 to 40 less the 5′ TTTN motif of said SEQ ID NO 1-40, or ii) the protein CAS13 and an RNA guide strand that comprises a sequence that is complimentary to one of SEQ ID NO 41 to 80, where the plasmid is adapted for expression of the protein and the transcription of the RNA guide strand in a mammalian cell; or
a therapeutically acceptable virus, that, when introduced into human cells, causes the expression of i) the protein Cpf1 and an RNA guide strand which comprises a sequence that is one of SEQ ID NO 1 to 40 less the 5′ TTTN motif of said SEQ ID NO 1-40, or ii) the protein CAS13 and an RNA guide strand that comprises a sequence that is complimentary to one of SEQ ID NO 41 to 80, or
a pharmaceutical according to claim 9,
to a patient in need thereof.

11. A method of causing double strand breaks in a HIV-infected cell comprising using a protein-RNA complex comprising the protein Cpf1 and an RNA guide strand which comprises a sequence that is one of SEQ ID NO 1 to 40 less the 5′ TTTN motif of said SEQ ID NO 1-40, where the method comprises introducing the protein-RNA complex, or means for expression thereof, in a cell.

12. A method for RNA knock-down of HIV RNA in a HIV-infected cell comprising using a protein-RNA complex comprising the CAS13 protein and an RNA guide strand that comprises a sequence that is complimentary to one of SEQ ID NO 41 to 80, and where the method comprises introducing the protein-RNA complex, or means for expression thereof, in a cell.

13. An RNA polynucleotide with a length of at most 100 nucleotides, the sequence comprising a Cpf1 handle sequence and an RNA sequence selected from one of SEQ ID NO 1 to SEQ ID NO 40 less the 5′TTTN motif.

14. An RNA polynucleotide with a length of at most 100 nucleotides comprising a CAS13 handle sequence, and a sequence that is complimentary to one of the sequences SEQ ID NO 41 to SEQ ID NO 80.


SEQUENCE LISTING (340 sequences)

SEQ ID NO: 1 - DNA, 24 bp, Human immunodeficiency virus 1
tttaaaagaaaaggggggattggg

SEQ ID NO: 2 - DNA, 24 bp, Human immunodeficiency virus 1
tttaaaagaaaaggggggactgga

SEQ ID NO: 3 - DNA, 24 bp, Human immunodeficiency virus 1
tttcccctgcactgtaccccccaa

SEQ ID NO: 4 - DNA, 24 bp, Human immunodeficiency virus 1
tttgtatgtctgttgctattatgt

SEQ ID NO: 5 - DNA, 24 bp, Human immunodeficiency virus 1
tttgactagcggaggctagaagga

SEQ ID NO: 6 - DNA, 24 bp, Human immunodeficiency virus 1
tttatataattcacttctccaatt

SEQ ID NO: 7 - DNA, 24 bp, Human immunodeficiency virus 1
tttcgggtttattacagggacagc

SEQ ID NO: 8 - DNA, 24 bp, Human immunodeficiency virus 1
tttatctacttgttcatttcctcc

SEQ ID NO: 9 - DNA, 24 bp, Human immunodeficiency virus 1
tttcttccaattatgttgacaggt

SEQ ID NO: 10 - DNA, 24 bp, Human immunodeficiency virus 1
tttattttttcttctgtcaatggc

SEQ ID NO: 11 - DNA, 24 bp, Human immunodeficiency virus 1
tttgataaaacctccaattccccc

SEQ ID NO: 12 - DNA, 24 bp, Human immunodeficiency virus 1
tttagtttgtatgtctgttgctat

SEQ ID NO: 13 - DNA, 24 bp, Human immunodeficiency virus 1
tttatatttatataattcacttct

SEQ ID NO: 14 - DNA, 24 bp, Human immunodeficiency virus 1
tttacccatgcatttaaagttcta

SEQ ID NO: 15 - DNA, 24 bp, Human immunodeficiency virus 1
tttaaaattgtgaatgaatactgc

SEQ ID NO: 16 - DNA, 24 bp, Human immunodeficiency virus 1
tttactggtacagtttcaatagga

SEQ ID NO: 17 - DNA, 24 bp, Human immunodeficiency virus 1
tttaaatcttgtggggtggctcct

SEQ ID NO: 18 - DNA, 24 bp, Human immunodeficiency virus 1
tttgaatttttgtaatttgttttt

SEQ ID NO: 19 - DNA, 24 bp, Human immunodeficiency virus 1
tttaattttactggtacagtttca

SEQ ID NO: 20 - DNA, 24 bp, Human immunodeficiency virus 1
tttcttctgtcaatggccattgtt

SEQ ID NO: 21 - DNA, 24 bp, Human immunodeficiency virus 1
tttcttttaaaattgtgaatgaat

SEQ ID NO: 22 - DNA, 24 bp, Human immunodeficiency virus 1
tttgggccatccattcctggcttt

SEQ ID NO: 23 - DNA, 24 bp, Human immunodeficiency virus 1
tttccacacaggtaccccataata

SEQ ID NO: 24 - DNA, 24 bp, Human immunodeficiency virus 1
tttcaggcccaatttttgaaattt

SEQ ID NO: 25 - DNA, 24 bp, Human immunodeficiency virus 1
tttatcaaagtaagacagtatgat

SEQ ID NO: 26 - DNA, 24 bp, Human immunodeficiency virus 1
tttccatccctgtggaagcacatt

SEQ ID NO: 27 - DNA, 24 bp, Human immunodeficiency virus 1
tttccataatccctaatgatcttt

SEQ ID NO: 28 - DNA, 24 bp, Human immunodeficiency virus 1
tttgcatggctgcttgatgtcccc

SEQ ID NO: 29 - DNA, 24 bp, Human immunodeficiency virus 1
tttccctaaaaaattagcctgtct

SEQ ID NO: 30 - DNA, 24 bp, Human immunodeficiency virus 1
tttgtttttgtaattctttagttt

SEQ ID NO: 31 - DNA, 24 bp, Human immunodeficiency virus 1
tttgtaattctttagtttgtatgt

SEQ ID NO: 32 - DNA, 24 bp, Human immunodeficiency virus 1
tttactggccatcttcctgctaat

SEQ ID NO: 33 - DNA, 24 bp, Human immunodeficiency virus 1
tttccacatgttaaaattttctgt

SEQ ID NO: 34 - DNA, 24 bp, Human immunodeficiency virus 1
tttgctggtcctttccaaagtgga

SEQ ID NO: 35 - DNA, 24 bp, Human immunodeficiency virus 1
tttggggttgctctggaaaactca

SEQ ID NO: 36 - DNA, 24 bp, Human immunodeficiency virus 1
tttgttcctgaagggtactagtag

SEQ ID NO: 37 - DNA, 24 bp, Human immunodeficiency virus 1
tttggatgggttatgaactccatc

SEQ ID NO: 38 - DNA, 24 bp, Human immunodeficiency virus 1
tttgctgttgcactataccagaca

SEQ ID NO: 39 - DNA, 24 bp, Human immunodeficiency virus 1
tttcaaaaattgggcctgaaaatc

SEQ ID NO: 40 - DNA, 24 bp, Human immunodeficiency virus 1
tttaacatttgcatggctgcttga

SEQ ID NO: 41 - RNA, 30 nt, Human immunodeficiency virus 1
cacaauuuuaaaagaaaaggggggauuggg

SEQ ID NO: 42 - RNA, 30 nt, Human immunodeficiency virus 1
acaauuuuaaaagaaaaggggggauugggg

SEQ ID NO: 43 - RNA, 30 nt, Human immunodeficiency virus 1
caauuuuaaaagaaaaggggggauuggggg

SEQ ID NO: 44 - RNA, 30 nt, Human immunodeficiency virus 1
uauggaaaacagauggcaggugaugauugu

SEQ ID NO: 45 - RNA, 30 nt, Human immunodeficiency virus 1
gaaaggugaaggggcaguaguaauacaaga

SEQ ID NO: 46 - RNA, 30 nt, Human immunodeficiency virus 1
ggaaaggugaaggggcaguaguaauacaag

SEQ ID NO: 47 - RNA, 30 nt, Human immunodeficiency virus 1
uggaaaggugaaggggcaguaguaauacaa

SEQ ID NO: 48 - RNA, 30 nt, Human immunodeficiency virus 1
aaaaggggggauugggggguacagugcagg

SEQ ID NO: 49 - RNA, 30 nt, Human immunodeficiency virus 1
gaaaaggggggauugggggguacagugcag

SEQ ID NO: 50 - RNA, 30 nt, Human immunodeficiency virus 1
agaaaaggggggauugggggguacagugca

SEQ ID NO: 51 - RNA, 30 nt, Human immunodeficiency virus 1
cucuggaaaggugaaggggcaguaguaaua

SEQ ID NO: 52 - RNA, 30 nt, Human immunodeficiency virus 1
cuggaaaggugaaggggcaguaguaauaca

SEQ ID NO: 53 - RNA, 30 nt, Human immunodeficiency virus 1
ucuggaaaggugaaggggcaguaguaauac

SEQ ID NO: 54 - RNA, 30 nt, Human immunodeficiency virus 1
aaacagauggcaggugaugauuguguggca

SEQ ID NO: 55 - RNA, 30 nt, Human immunodeficiency virus 1
aggggggauugggggguacagugcagggga

SEQ ID NO: 56 - RNA, 30 nt, Human immunodeficiency virus 1
aaaacagauggcaggugaugauuguguggc

SEQ ID NO: 57 - RNA, 30 nt, Human immunodeficiency virus 1
gaaaacagauggcaggugaugauugugugg

SEQ ID NO: 58 - RNA, 30 nt, Human immunodeficiency virus 1
ggaaaacagauggcaggugaugauugugug

SEQ ID NO: 59 - RNA, 30 nt, Human immunodeficiency virus 1
auggaaaacagauggcaggugaugauugug

SEQ ID NO: 60 - RNA, 30 nt, Human immunodeficiency virus 1
uggaaaacagauggcaggugaugauugugu

SEQ ID NO: 61 - RNA, 30 nt, Human immunodeficiency virus 1
aauuuuaaaagaaaaggggggauugggggg

SEQ ID NO: 62 - RNA, 30 nt, Human immunodeficiency virus 1
auuuuaaaagaaaaggggggauuggggggu

SEQ ID NO: 63 - RNA, 30 nt, Human immunodeficiency virus 1
uuuuaaaagaaaaggggggauuggggggua

SEQ ID NO: 64 - RNA, 30 nt, Human immunodeficiency virus 1
aaaaauucaaaauuuucggguuuauuacag

SEQ ID NO: 65 - RNA, 30 nt, Human immunodeficiency virus 1
uagaaggagagagaugggugcgagagcguc

SEQ ID NO: 66 - RNA, 30 nt, Human immunodeficiency virus 1
cuagaaggagagagaugggugcgagagcgu

SEQ ID NO: 67 - RNA, 30 nt, Human immunodeficiency virus 1
gcuagaaggagagagaugggugcgagagcg

SEQ ID NO: 68 - RNA, 30 nt, Human immunodeficiency virus 1
ggcuagaaggagagagaugggugcgagagc

SEQ ID NO: 69 - RNA, 30 nt, Human immunodeficiency virus 1
aaaggggggauugggggguacagugcaggg

SEQ ID NO: 70 - RNA, 30 nt, Human immunodeficiency virus 1
aaggggggauugggggguacagugcagggg

SEQ ID NO: 71 - RNA, 30 nt, Human immunodeficiency virus 1
uuaaaagaaaaggggggauugggggguaca

SEQ ID NO: 72 - RNA, 30 nt, Human immunodeficiency virus 1
uuuaaaagaaaaggggggauugggggguac

SEQ ID NO: 73 - RNA, 30 nt, Human immunodeficiency virus 1
ucuugggagcagcaggaagcacuaugggcg

SEQ ID NO: 74 - RNA, 30 nt, Human immunodeficiency virus 1
aggcuagaaggagagagaugggugcgagag

SEQ ID NO: 75 - RNA, 30 nt, Human immunodeficiency virus 1
uucuugggagcagcaggaagcacuaugggc

SEQ ID NO: 76 - RNA, 30 nt, Human immunodeficiency virus 1
auuauggaaaacagauggcaggugaugauu

SEQ ID NO: 77 - RNA, 30 nt, Human immunodeficiency virus 1
cuugggagcagcaggaagcacuaugggcgc

SEQ ID NO: 78 - RNA, 30 nt, Human immunodeficiency virus 1
uuauggaaaacagauggcaggugaugauug

SEQ ID NO: 79 - RNA, 30 nt, Human immunodeficiency virus 1
aaaagaaaaggggggauugggggguacagu

SEQ ID NO: 80 - RNA, 30 nt, Human immunodeficiency virus 1
uaaaagaaaaggggggauugggggguacag

SEQ ID NO: 81 - skipped

SEQ ID NO: 82 - DNA, 20 bp, Human immunodeficiency virus 1
aaagaaaaggggggattggg

SEQ ID NO: 83 - RNA, 20 nt, Human immunodeficiency virus 1
aaagaaaaggggggauuggg

SEQ ID NO: 84 - RNA, 20 nt, Lachnospiraceae
uaauuucuacucuuguagau

SEQ ID NO: 85 - RNA, 40 nt, synthetic organisms
  Example guide strand
uaauuucuacucuuguagauaaagaaaaggggggauuggg

SEQ ID NO: 86 - RNA, 20 nt, Acidaminococcus
  Cpf1 guide strand handle sequence
aauuucuacucuuguagaug

SEQ ID NO: 87 - skipped

SEQ ID NO: 88 - RNA, 30 nt, Human immunodeficiency virus 1
cccaauccccccuuuucuuuuaaaauugug

SEQ ID NO: 89 - RNA, 36 nt, Prevotella
guuguggaagguccaguuuugaggggcuauuacaac

SEQ ID NO: 90 - RNA, 36 nt, Leptotrichia
gauuuagacuaccccaaaaacgaaggggacuaaaac

SEQ ID NO: 91 - RNA, 66 nt, synthetic organisms
gauuuagacuaccccaaaaacgaaggggacuaaaaccccaauccccccuuuucuuuuaaaauugug

SEQ ID NO: 92-100 - skipped (9 entries)

SEQ ID NO: 101 - DNA, 24 bp, Hepatitis B virus
tttactagtgccatttgttcagtg

SEQ ID NO: 102 - DNA, 24 bp, Hepatitis B virus
tttgttcagtggttcgtagggctt

SEQ ID NO: 103 - DNA, 24 bp, Hepatitis B virus
tttcttttgtctttgggtatacat

SEQ ID NO: 104 - DNA, 24 bp, Hepatitis B virus
tttgaagtatgcctcaaggtcggt

SEQ ID NO: 105 - DNA, 24 bp, Hepatitis B virus
tttctcgccaacttacaaggcctt

SEQ ID NO: 106 - DNA, 24 bp, Hepatitis B virus
tttcagttatatggatgatgtggt

SEQ ID NO: 107 - DNA, 24 bp, Hepatitis B virus
tttccggaagtgttgataagatag

SEQ ID NO: 108 - DNA, 24 bp, Hepatitis B virus
tttgccttctgacttctttccttc

SEQ ID NO: 109 - DNA, 24 bp, Hepatitis B virus
tttggagtgtggattcgcactcct

SEQ ID NO: 110 - DNA, 24 bp, Hepatitis B virus
tttatgcctacagcctcctagtac

SEQ ID NO: 111 - DNA, 24 bp, Hepatitis B virus
tttgtactaggaggctgtaggcat

SEQ ID NO: 112 - DNA, 24 bp, Hepatitis B virus
tttcccccactgtttggctttcag

SEQ ID NO: 113 - DNA, 24 bp, Hepatitis B virus
tttcttgttgacaagaatcctcac

SEQ ID NO: 114 - DNA, 24 bp, Hepatitis B virus
tttggtgtcttttggagtgtggat

SEQ ID NO: 115 - DNA, 24 bp, Hepatitis B virus
tttggggtggagccctcaggctca

SEQ ID NO: 116 - DNA, 24 bp, Hepatitis B virus
tttggagcttctgtggagttactc

SEQ ID NO: 117 - DNA, 24 bp, Hepatitis B virus
tttaaatgtatacccaaagacaaa

SEQ ID NO: 118 - DNA, 24 bp, Hepatitis B virus
tttgtctttgggtatacatttaaa

SEQ ID NO: 119 - DNA, 24 bp, Hepatitis B virus
tttgtggctcctctgccgatccat

SEQ ID NO: 120 - DNA, 24 bp, Hepatitis B virus
tttacgtcccgtcggcgctgaatc

SEQ ID NO: 121 - DNA, 24 bp, Hepatitis B virus
tttctcctggctcagtttactagt

SEQ ID NO: 122 - DNA, 24 bp, Hepatitis B virus
tttgtttacgtcccgtcggcgctg

SEQ ID NO: 123 - DNA, 24 bp, Hepatitis B virus
tttgtgggtcaccatattcttggg

SEQ ID NO: 124 - DNA, 24 bp, Hepatitis B virus
tttggaagaccaacctcccatgct

SEQ ID NO: 125 - DNA, 24 bp, Hepatitis B virus
tttcacatttcctgtcttactttt

SEQ ID NO: 126 - DNA, 24 bp, Hepatitis B virus
tttacgcggtctccccgtctgtgc

SEQ ID NO: 127 - DNA, 24 bp, Hepatitis B virus
tttcacctctgcctaatcatctca

SEQ ID NO: 128 - DNA, 24 bp, Hepatitis B virus
tttcactttctcgccaacttacaa

SEQ ID NO: 129 - DNA, 24 bp, Hepatitis B virus
tttggctttcagttatatggatga

SEQ ID NO: 130 - DNA, 24 bp, Hepatitis B virus
tttccatggctgctaggctgtgct

SEQ ID NO: 131 - DNA, 24 bp, Hepatitis B virus
tttatcatattcctcttcatcctg

SEQ ID NO: 132 - DNA, 24 bp, Hepatitis B virus
tttatacgggtcaatgtccatgcc

SEQ ID NO: 133 - DNA, 24 bp, Hepatitis B virus
tttgctgccccttttacacaatgt

SEQ ID NO: 134 - DNA, 24 bp, Hepatitis B virus
tttacacagaaaggccttgtaagt

SEQ ID NO: 135 - DNA, 24 bp, Hepatitis B virus
tttacgcggactccccgtctgtgc

SEQ ID NO: 136 - DNA, 24 bp, Hepatitis B virus
tttgtcctggttatcgctggatgt

SEQ ID NO: 137 - DNA, 24 bp, Hepatitis B virus
tttattcttctactgtacctgtct

SEQ ID NO: 138 - DNA, 24 bp, Hepatitis B virus
tttcacctctgcctaatcatctct

SEQ ID NO: 139 - DNA, 24 bp, Hepatitis B virus
tttctcttggctcagtttactagt

SEQ ID NO: 140 - DNA, 24 bp, Hepatitis B virus
tttcctgtcttacttttggaagag

SEQ ID NO: 141 - RNA, 30 nt, Hepatitis B virus
ccgugugcacuucgcuucaccucugcacgu

SEQ ID NO: 142 - RNA, 30 nt, Hepatitis B virus
agagucuagacucgugguggacuucucuca

SEQ ID NO: 143 - RNA, 30 nt, Hepatitis B virus
cagagucuagacucgugguggacuucucuc

SEQ ID NO: 144 - RNA, 30 nt, Hepatitis B virus
uucaagccuccaagcugugccuuggguggc

SEQ ID NO: 145 - RNA, 30 nt, Hepatitis B virus
caagccuccaagcugugccuuggguggcuu

SEQ ID NO: 146 - RNA, 30 nt, Hepatitis B virus
ucaagccuccaagcugugccuuggguggcu

SEQ ID NO: 147 - RNA, 30 nt, Hepatitis B virus
aagccuccaagcugugccuuggguggcuuu

SEQ ID NO: 148 - RNA, 30 nt, Hepatitis B virus
acucgugguggacuucucucaauuuucuag

SEQ ID NO: 149 - RNA, 30 nt, Hepatitis B virus
cgugguggacuucucucaauuuucuagggg

SEQ ID NO: 150 - RNA, 30 nt, Hepatitis B virus
ucgugguggacuucucucaauuuucuaggg

SEQ ID NO: 151 - RNA, 30 nt, Hepatitis B virus
cucgugguggacuucucucaauuuucuagg

SEQ ID NO: 152 - RNA, 30 nt, Hepatitis B virus
gacucgugguggacuucucucaauuuucua

SEQ ID NO: 153 - RNA, 30 nt, Hepatitis B virus
cuagacucgugguggacuucucucaauuuu

SEQ ID NO: 154 - RNA, 30 nt, Hepatitis B virus
uagacucgugguggacuucucucaauuuuc

SEQ ID NO: 155 - RNA, 30 nt, Hepatitis B virus
ucuagacucgugguggacuucucucaauuu

SEQ ID NO: 156 - RNA, 30 nt, Hepatitis B virus
agacucgugguggacuucucucaauuuucu

SEQ ID NO: 157 - RNA, 30 nt, Hepatitis B virus
agucuagacucgugguggacuucucucaau

SEQ ID NO: 158 - RNA, 30 nt, Hepatitis B virus
gucuagacucgugguggacuucucucaauu

SEQ ID NO: 159 - RNA, 30 nt, Hepatitis B virus
gagucuagacucgugguggacuucucucaa

SEQ ID NO: 160 - RNA, 30 nt, Hepatitis B virus
ugcaacuuuuucaccucugccuaaucaucu

SEQ ID NO: 161 - RNA, 30 nt, Hepatitis B virus
guucaagccuccaagcugugccuugggugg

SEQ ID NO: 162 - RNA, 30 nt, Hepatitis B virus
uguucaagccuccaagcugugccuugggug

SEQ ID NO: 163 - RNA, 30 nt, Hepatitis B virus
acuguucaagccuccaagcugugccuuggg

SEQ ID NO: 164 - RNA, 30 nt, Hepatitis B virus
cuguucaagccuccaagcugugccuugggu

SEQ ID NO: 165 - RNA, 30 nt, Hepatitis B virus
uagaagaagaacucccucgccucgcagacg

SEQ ID NO: 166 - RNA, 30 nt, Hepatitis B virus
caugcaacuuuuucaccucugccuaaucau

SEQ ID NO: 167 - RNA, 30 nt, Hepatitis B virus
augcaacuuuuucaccucugccuaaucauc

SEQ ID NO: 168 - RNA, 30 nt, Hepatitis B virus
cuagaagaagaacucccucgccucgcagac

SEQ ID NO: 169 - RNA, 30 nt, Hepatitis B virus
ccuagaagaagaacucccucgccucgcaga

SEQ ID NO: 170 - RNA, 30 nt, Hepatitis B virus
cccuagaagaagaacucccucgccucgcag

SEQ ID NO: 171 - RNA, 30 nt, Hepatitis B virus
ccccuagaagaagaacucccucgccucgca

SEQ ID NO: 172 - RNA, 30 nt, Hepatitis B virus
ucaguuuacuagugccauuuguucaguggu

SEQ ID NO: 173 - RNA, 30 nt, Hepatitis B virus
uggcucaguuuacuagugccauuuguucag

SEQ ID NO: 174 - RNA, 30 nt, Hepatitis B virus
ggcucaguuuacuagugccauuuguucagu

SEQ ID NO: 175 - RNA, 30 nt, Hepatitis B virus
guuuacuagugccauuuguucagugguucg

SEQ ID NO: 176 - RNA, 30 nt, Hepatitis B virus
aguuuacuagugccauuuguucagugguuc

SEQ ID NO: 177 - RNA, 30 nt, Hepatitis B virus
caguuuacuagugccauuuguucagugguu

SEQ ID NO: 178 - RNA, 30 nt, Hepatitis B virus
cucaguuuacuagugccauuuguucagugg

SEQ ID NO: 179 - RNA, 30 nt, Hepatitis B virus
gcucaguuuacuagugccauuuguucagug

SEQ ID NO: 180 - RNA, 30 nt, Hepatitis B virus
ccaugcaacuuuuucaccucugccuaauca

SEQ ID NO: 181 - DNA, 24 bp, Human herpesvirus 1
tttaataaaaataaccaaaaacac

SEQ ID NO: 182 - DNA, 24 bp, Human herpesvirus 1
tttgtttgaaatgttttgttttta

SEQ ID NO: 183 - DNA, 24 bp, Human herpesvirus 1
tttgggtgggtggggagtgggtgg

SEQ ID NO: 184 - DNA, 24 bp, Human herpesvirus 1
tttgaaatgttttgtttttattgt

SEQ ID NO: 185 - DNA, 24 bp, Human herpesvirus 1
tttaggatgccagccagggcggcg

SEQ ID NO: 186 - DNA, 24 bp, Human herpesvirus 1
tttatacacagatgtcaacgccgc

SEQ ID NO: 187 - DNA, 24 bp, Human herpesvirus 1
tttggaaatagcaacaaggccgtg

SEQ ID NO: 188 - DNA, 24 bp, Human herpesvirus 1
tttggatggtatggtccagatgct

SEQ ID NO: 189 - DNA, 24 bp, Human herpesvirus 1
tttctgacctgcaccgatcgcagc

SEQ ID NO: 190 - DNA, 24 bp, Human herpesvirus 1
tttagttctatgatgacacaaacc

SEQ ID NO: 191 - DNA, 24 bp, Human herpesvirus 1
tttggcgtacatgttttgggccgc

SEQ ID NO: 192 - DNA, 24 bp, Human herpesvirus 1
tttctgtttctttaacccgtctgg

SEQ ID NO: 193 - DNA, 24 bp, Human herpesvirus 1
tttaggccgcaacccggccgagcc

SEQ ID NO: 194 - DNA, 24 bp, Human herpesvirus 1
tttcggtacagcaggtagagacac

SEQ ID NO: 195 - DNA, 24 bp, Human herpesvirus 1
tttgagcagatgtttaccgatgcc

SEQ ID NO: 196 - DNA, 24 bp, Human herpesvirus 1
tttctacctccccggggcctgcat

SEQ ID NO: 197 - DNA, 24 bp, Human herpesvirus 1
tttgcttccccggacttcgcgccc

SEQ ID NO: 198 - DNA, 24 bp, Human herpesvirus 1
tttgactcagacgcagggcccggg

SEQ ID NO: 199 - DNA, 24 bp, Human herpesvirus 1
tttaaaaagatatacagtaagaca

SEQ ID NO: 200 - DNA, 24 bp, Human herpesvirus 1
tttcactgggagcgctttccggac

SEQ ID NO: 201 - DNA, 24 bp, Human herpesvirus 1
tttgggtaaacacctttaataagc

SEQ ID NO: 202 - DNA, 24 bp, Human herpesvirus 1
tttaattaccataccgggaagtgg

SEQ ID NO: 203 - DNA, 24 bp, Human herpesvirus 1
tttgccgggacggtgaccgggcga

SEQ ID NO: 204 - DNA, 24 bp, Human herpesvirus 1
tttacggatcggtgtataaattac

SEQ ID NO: 205 - DNA, 24 bp, Human herpesvirus 1
tttggggcgtgtctgtttcttggc

SEQ ID NO: 206 - DNA, 24 bp, Human herpesvirus 1
tttctgtcgtcggaggcccccggg

SEQ ID NO: 207 - DNA, 24 bp, Human herpesvirus 1
tttgggccgcaatgcgcgtggcgc

SEQ ID NO: 208 - DNA, 24 bp, Human herpesvirus 1
tttggcctgacggaaaggcttcgc

SEQ ID NO: 209 - DNA, 24 bp, Human herpesvirus 1
tttgacttgcacgaactcgctgac

SEQ ID NO: 210 - DNA, 24 bp, Human herpesvirus 1
tttggggattggcggccaggcccg

SEQ ID NO: 211 - DNA, 24 bp, Human herpesvirus 1
tttgccagcaacctgaccgcgctg

SEQ ID NO: 212 - DNA, 24 bp, Human herpesvirus 1
tttgaaactgacatcgcgataccc

SEQ ID NO: 213 - DNA, 24 bp, Human herpesvirus 1
tttgcatcggagcgcacgcgggaa

SEQ ID NO: 214 - DNA, 24 bp, Human herpesvirus 1
tttctggatggccgacatttcccc

SEQ ID NO: 215 - DNA, 24 bp, Human herpesvirus 1
tttcgttttctcccccgaagtcag

SEQ ID NO: 216 - DNA, 24 bp, Human herpesvirus 1
tttgggcgcctccggatcaccaac

SEQ ID NO: 217 - DNA, 24 bp, Human herpesvirus 1
tttcctcatggccccttttatacc

SEQ ID NO: 218 - DNA, 24 bp, Human herpesvirus 1
tttggggaggggaaaggcgtgggg

SEQ ID NO: 219 - DNA, 24 bp, Human herpesvirus 1
tttgcgtggccgcctcgtaaaacc

SEQ ID NO: 220 - DNA, 24 bp, Human herpesvirus 1
tttaaaaaggcctcggccctccct

SEQ ID NO: 221 - RNA, 30 nt, Human herpesvirus 1
uggguggggaguggguggguggggaguggg

SEQ ID NO: 222 - RNA, 30 nt, Human herpesvirus 1
uuuuuggguggguggggagugggugggugg

SEQ ID NO: 223 - RNA, 30 nt, Human herpesvirus 1
aguggguggguggggagugggugggugggg

SEQ ID NO: 224 - RNA, 30 nt, Human herpesvirus 1
uugauuuuuggguggguggggagugggugg

SEQ ID NO: 225 - RNA, 30 nt, Human herpesvirus 1
ugauuuuuggguggguggggaguggguggg

SEQ ID NO: 226 - RNA, 30 nt, Human herpesvirus 1
uuuuggguggguggggaguggguggguggg

SEQ ID NO: 227 - RNA, 30 nt, Human herpesvirus 1
gaguggguggguggggaguggguggguggg

SEQ ID NO: 228 - RNA, 30 nt, Human herpesvirus 1
auaauguaauugguggaugagaaguaggug

SEQ ID NO: 229 - RNA, 30 nt, Human herpesvirus 1
gguggguggggaguggguggguggggagug

SEQ ID NO: 230 - RNA, 30 nt, Human herpesvirus 1
uuggguggguggggagugggugggugggga

SEQ ID NO: 231 - RNA, 30 nt, Human herpesvirus 1
guggguggggaguggguggguggggagugg

SEQ ID NO: 232 - RNA, 30 nt, Human herpesvirus 1
uaauguaauugguggaugagaaguagguga

SEQ ID NO: 233 - RNA, 30 nt, Human herpesvirus 1
uggguggguggggaguggguggguggggag

SEQ ID NO: 234 - RNA, 30 nt, Human herpesvirus 1
uuuggguggguggggagugggugggugggg

SEQ ID NO: 235 - RNA, 30 nt, Human herpesvirus 1
ggaguggguggguggggagugggugggugg

SEQ ID NO: 236 - RNA, 30 nt, Human herpesvirus 1
auuuuuggguggguggggagugggugggug

SEQ ID NO: 237 - RNA, 30 nt, Human herpesvirus 1
gggaguggguggguggggagugggugggug

SEQ ID NO: 238 - RNA, 30 nt, Human herpesvirus 1
gauuuuuggguggguggggagugggugggu

SEQ ID NO: 239 - RNA, 30 nt, Human herpesvirus 1
gguggggaguggguggguggggagugggug

SEQ ID NO: 240 - RNA, 30 nt, Human herpesvirus 1
ggggaguggguggguggggagugggugggu

SEQ ID NO: 241 - RNA, 30 nt, Human herpesvirus 1
ggguggguggggaguggguggguggggagu

SEQ ID NO: 242 - RNA, 30 nt, Human herpesvirus 1
ggguggggaguggguggguggggagugggu

SEQ ID NO: 243 - RNA, 30 nt, Human herpesvirus 1
gguugauuuuuggguggguggggagugggu

SEQ ID NO: 244 - RNA, 30 nt, Human herpesvirus 1
guggggaguggguggguggggagugggugg

SEQ ID NO: 245 - RNA, 30 nt, Human herpesvirus 1
guggguggguggggagugggugggugggga

SEQ ID NO: 246 - RNA, 30 nt, Human herpesvirus 1
guugauuuuuggguggguggggagugggug

SEQ ID NO: 247 - RNA, 30 nt, Human herpesvirus 1
uggggaguggguggguggggaguggguggg

SEQ ID NO: 248 - RNA, 30 nt, Human herpesvirus 1
gggggggagaggggagagggggggagaggg

SEQ ID NO: 249 - RNA, 30 nt, Human herpesvirus 1
gggggagaggggagagggggggagagggga

SEQ ID NO: 250 - RNA, 30 nt, Human herpesvirus 1
ggggagagggggggagaggggagagggggg

SEQ ID NO: 251 - RNA, 30 nt, Human herpesvirus 1
ggggggagaggggagagggggggagagggg

SEQ ID NO: 252 - RNA, 30 nt, Human herpesvirus 1
gggagagggggggagaggggagaggggggg

SEQ ID NO: 253 - RNA, 30 nt, Human herpesvirus 1
gagggggggagaggggagagggggggagag

SEQ ID NO: 254 - RNA, 30 nt, Human herpesvirus 1
aggggagagggggggagaggggagaggggg

SEQ ID NO: 255 - RNA, 30 nt, Human herpesvirus 1
agagggggggagaggggagagggggggaga

SEQ ID NO: 256 - RNA, 30 nt, Human herpesvirus 1
agggggggagaggggagagggggggagagg

SEQ ID NO: 257 - RNA, 30 nt, Human herpesvirus 1
ggagagggggggagaggggagaggggggga

SEQ ID NO: 258 - RNA, 30 nt, Human herpesvirus 1
gagagggggggagaggggagagggggggag

SEQ ID NO: 259 - RNA, 30 nt, Human herpesvirus 1
ggggagaggggagagggggggagaggggag

SEQ ID NO: 260 - RNA, 30 nt, Human herpesvirus 1
aaaaaagggagggacgggggccggcagacc

SEQ ID NO: 261 - DNA, 24 bp, Human herpesvirus 2
tttctccttcctcttcccttccac

SEQ ID NO: 262 - DNA, 24 bp, Human herpesvirus 2
tttatatttataaaaattttacaa

SEQ ID NO: 263 - DNA, 24 bp, Human herpesvirus 2
tttattgtaaaatttttataaata

SEQ ID NO: 264 - DNA, 24 bp, Human herpesvirus 2
tttaccctcaccccaccccatcct

SEQ ID NO: 265 - DNA, 24 bp, Human herpesvirus 2
tttattattaattacaccaaccac

SEQ ID NO: 266 - DNA, 24 bp, Human herpesvirus 2
tttcatatattttaaataaacaaa

SEQ ID NO: 267 - DNA, 24 bp, Human herpesvirus 2
tttgtttatttaaaatatatgaaa

SEQ ID NO: 268 - DNA, 24 bp, Human herpesvirus 2
tttataattcttttttatttccca

SEQ ID NO: 269 - DNA, 24 bp, Human herpesvirus 2
tttatacacaacaccaacctttcc

SEQ ID NO: 270 - DNA, 24 bp, Human herpesvirus 2
tttccaccccccttccccctcctt

SEQ ID NO: 271 - DNA, 24 bp, Human herpesvirus 2
tttattttatacacaacaccaacc

SEQ ID NO: 272 - DNA, 24 bp, Human herpesvirus 2
tttgtgtttatttaaggagaaggg

SEQ ID NO: 273 - DNA, 24 bp, Human herpesvirus 2
tttgttggatgatggattgattga

SEQ ID NO: 274 - DNA, 24 bp, Human herpesvirus 2
tttggggggggtgtttgggtggga

SEQ ID NO: 275 - DNA, 24 bp, Human herpesvirus 2
tttctggccttgttgaaaacttga

SEQ ID NO: 276 - DNA, 24 bp, Human herpesvirus 2
tttgacggcgccggggttgaagcg

SEQ ID NO: 277 - DNA, 24 bp, Human herpesvirus 2
tttatcgccgcggctgcgccctgc

SEQ ID NO: 278 - DNA, 24 bp, Human herpesvirus 2
tttatgaagtacgtcgagtggtcg

SEQ ID NO: 279 - DNA, 24 bp, Human herpesvirus 2
tttggcccccaccagagcgagtgg

SEQ ID NO: 280 - DNA, 24 bp, Human herpesvirus 2
tttaactttggggatttcggcgac

SEQ ID NO: 281 - DNA, 24 bp, Human herpesvirus 2
tttcctgggtgtcggccggaaaca

SEQ ID NO: 282 - DNA, 24 bp, Human herpesvirus 2
tttcacgtaggcgaacatgctgtc

SEQ ID NO: 283 - DNA, 24 bp, Human herpesvirus 2
tttcggggcctcgacaaggaggcg

SEQ ID NO: 284 - DNA, 24 bp, Human herpesvirus 2
tttgtttcgtgcgtttgggtgcgg

SEQ ID NO: 285 - DNA, 24 bp, Human herpesvirus 2
tttaattaccataagcgggaatgg

SEQ ID NO: 286 - DNA, 24 bp, Human herpesvirus 2
tttccggagttagcaaaaccgata

SEQ ID NO: 287 - DNA, 24 bp, Human herpesvirus 2
tttccgccctcctccctccccacc

SEQ ID NO: 288 - DNA, 24 bp, Human herpesvirus 2
tttaccagcgccccctcgccgacg

SEQ ID NO: 289 - DNA, 24 bp, Human herpesvirus 2
tttcgagacgggcgcatgtccaag

SEQ ID NO: 290 - DNA, 24 bp, Human herpesvirus 2
tttatcggttggacgcgtttccct

SEQ ID NO: 291 - DNA, 24 bp, Human herpesvirus 2
tttccgcgcggctgggtgagcgtg

SEQ ID NO: 292 - DNA, 24 bp, Human herpesvirus 2
tttcctggccctcaagcaggggcc

SEQ ID NO: 293 - DNA, 24 bp, Human herpesvirus 2
tttcccccgcctcccgtcttcttc

SEQ ID NO: 294 - DNA, 24 bp, Human herpesvirus 2
tttcacggcgacgatccgtttggg

SEQ ID NO: 295 - DNA, 24 bp, Human herpesvirus 2
tttcattatcttcgccctggagca

SEQ ID NO: 296 - DNA, 24 bp, Human herpesvirus 2
tttcgccgaaccccatggcctcga

SEQ ID NO: 297 - DNA, 24 bp, Human herpesvirus 2
tttgttgtatgtaacctgaccgga

SEQ ID NO: 298 - DNA, 24 bp, Human herpesvirus 2
tttggtccttttctctggtttcgg

SEQ ID NO: 299 - DNA, 24 bp, Human herpesvirus 2
tttatacgcggaccccagcaccac

SEQ ID NO: 300 - DNA, 24 bp, Human herpesvirus 2
tttccaagcgggtggtgatgtttg

SEQ ID NO: 301 - RNA, 30 nt, Human herpesvirus 2
guggguggaagggaagaggaaggagaaagg

SEQ ID NO: 302 - RNA, 30 nt, Human herpesvirus 2
gguggguggaagggaagaggaaggagaaag

SEQ ID NO: 303 - RNA, 30 nt, Human herpesvirus 2
uguaaaauuuuuauaaauauaaaguuuuuu

SEQ ID NO: 304 - RNA, 30 nt, Human herpesvirus 2
gguggaagggaagaggaaggagaaaggggg

SEQ ID NO: 305 - RNA, 30 nt, Human herpesvirus 2
uggguggaagggaagaggaaggagaaaggg

SEQ ID NO: 306 - RNA, 30 nt, Human herpesvirus 2
guaaaauuuuuauaaauauaaaguuuuuuu

SEQ ID NO: 307 - RNA, 30 nt, Human herpesvirus 2
gggggaagagagggggagguagggagggga

SEQ ID NO: 308 - RNA, 30 nt, Human herpesvirus 2
gaagagagggggagguagggaggggagagg

SEQ ID NO: 309 - RNA, 30 nt, Human herpesvirus 2
uuguaaaauuuuuauaaauauaaaguuuuu

SEQ ID NO: 310 - RNA, 30 nt, Human herpesvirus 2
ggguggaagggaagaggaaggagaaagggg

SEQ ID NO: 311 - RNA, 30 nt, Human herpesvirus 2
guggaagggaagaggaaggagaaagggggg

SEQ ID NO: 312 - RNA, 30 nt, Human herpesvirus 2
uauuguaaaauuuuuauaaauauaaaguuu

SEQ ID NO: 313 - RNA, 30 nt, Human herpesvirus 2
uuauuguaaaauuuuuauaaauauaaaguu

SEQ ID NO: 314 - RNA, 30 nt, Human herpesvirus 2
uaaaauuuuuauaaauauaaaguuuuuuuu

SEQ ID NO: 315 - RNA, 30 nt, Human herpesvirus 2
uuuauuguaaaauuuuuauaaauauaaagu

SEQ ID NO: 316 - RNA, 30 nt, Human herpesvirus 2
ggaagagagggggagguagggaggggagag

SEQ ID NO: 317 - RNA, 30 nt, Human herpesvirus 2
gggaagagagggggagguagggaggggaga

SEQ ID NO: 318 - RNA, 30 nt, Human herpesvirus 2
ggggaagagagggggagguagggaggggag

SEQ ID NO: 319 - RNA, 30 nt, Human herpesvirus 2
ggguggguggaagggaagaggaaggagaaa

SEQ ID NO: 320 - RNA, 30 nt, Human herpesvirus 2
auuguaaaauuuuuauaaauauaaaguuuu

SEQ ID NO: 321 - RNA, 30 nt, Human herpesvirus 2
ggggguggguggaagggaagaggaaggaga

SEQ ID NO: 322 - RNA, 30 nt, Human herpesvirus 2
gggguggguggaagggaagaggaaggagaa

SEQ ID NO: 323 - RNA, 30 nt, Human herpesvirus 2
ugauguaauugguggaugagaaguagguga

SEQ ID NO: 324 - RNA, 30 nt, Human herpesvirus 2
ugggagguggguguuuguaugugugggaga

SEQ ID NO: 325 - RNA, 30 nt, Human herpesvirus 2
augauguaauugguggaugagaaguaggug

SEQ ID NO: 326 - RNA, 30 nt, Human herpesvirus 2
uguuggaugauggauugauugauuuuauug

SEQ ID NO: 327 - RNA, 30 nt, Human herpesvirus 2
uauuuuguuggaugauggauugauugauuu

SEQ ID NO: 328 - RNA, 30 nt, Human herpesvirus 2
guuggaugauggauugauugauuuuauuga

SEQ ID NO: 329 - RNA, 30 nt, Human herpesvirus 2
uuauuuuguuggaugauggauugauugauu

SEQ ID NO: 330 - RNA, 30 nt, Human herpesvirus 2
gggggagguagggaggggagaggagaaggg

SEQ ID NO: 331 - RNA, 30 nt, Human herpesvirus 2
gggggaggaaaaagaauaaaggggguagug

SEQ ID NO: 332 - RNA, 30 nt, Human herpesvirus 2
ggggggaggaaaaagaauaaaggggguagu

SEQ ID NO: 333 - RNA, 30 nt, Human herpesvirus 2
gauaugugaguuugguuguguuuuguggga

SEQ ID NO: 334 - RNA, 30 nt, Human herpesvirus 2
gagagggggagguagggaggggagaggaga

SEQ ID NO: 335 - RNA, 30 nt, Human herpesvirus 2
gagggggagguagggaggggagaggagaag

SEQ ID NO: 336 - RNA, 30 nt, Human herpesvirus 2
agggggagguagggaggggagaggagaagg

SEQ ID NO: 337 - RNA, 30 nt, Human herpesvirus 2
agagagggggagguagggaggggagaggag

SEQ ID NO: 338 - RNA, 30 nt, Human herpesvirus 2
aagagagggggagguagggaggggagagga

SEQ ID NO: 339 - RNA, 30 nt, Human herpesvirus 2
agagggggagguagggaggggagaggagaa

SEQ ID NO: 340 - RNA, 30 nt, Human herpesvirus 2
auuuuguuggaugauggauugauugauuuu